(19) United States
(12) Patent Application Publication
(10) Pub. No.: US 2010/0266594 A1
(43) Pub. Date: Oct. 21, 2010

(54) IL-9 IN FIBROTIC AND INFLAMMATORY DISEASE

(75) Inventor: Jennifer Reed, Clarksburg, MD (US)

Correspondence Address: MEDIMMUNE, LLC;Patrick Scott Alban, ONE MEDIMMUNE WAY, GAITHERSBURG, MD, 20878, US

(73) Assignee: MEDIMMUNE, LLC, Gaithersburg, MD (US)

(21) Appl. No.: 12/600,583

(22) PCT Filed: May 19, 2008
(86) PCT No.: PCT/US08/64049

§ 371 Date: May 10, 2010

Related U.S. Application Data
(60) Provisional application No. 60/924,541, filed on May 18, 2007.

(51) Int. Cl. A61K 39/395; A61P 1/00; A61P 11/00; A61P 19/00

(52) U.S. Cl. 424/135.1; 424/158.1; 424/145.1; 424/133.1

(57) ABSTRACT

The present invention encompasses methods of employing inhibitors of IL-9 in the treatment of fibrotic disorders, e.g., COPD and inflammatory bowel disease.

### FIELD OF THE INVENTION

The present invention relates to IL-9 and inflammatory and fibrotic diseases or disorders.

### BACKGROUND OF THE INVENTION

The present invention encompasses methods which employ IL-9 inhibitors to treat fibrotic and inflammatory diseases or disorders. These methods include treatment of subjects or patients having a fibrotic disease or disorder by administering IL-9 inhibitors, e.g., an antibody specific for IL-9.

### SUMMARY OF THE INVENTION

One embodiment of the invention encompasses a method of treating a fibrotic disorder. An IL-9 inhibitor is administered to a subject.

Other embodiments of the invention encompass methods of treating COPD or inflammatory bowel disease. An IL-9 inhibitor is administered to a subject.

### BRIEF DESCRIPTION OF THE FIGURES

FIGS. 1a and 1b: Immunohistochemistry analysis of resected human colon (non-specific colitis) samples using (a) IL-9 receptor antibody as primary antibody and (b) no primary antibody (negative control).

FIG. 2a-2c: Immunohistochemistry analysis of paraffin sections of resected human COPD lung tissue using (a and c) IL-9 receptor antibody as primary antibody and (b) no primary antibody (negative control).

FIG. 3: Immunohistochemistry analysis of cryo sections of resected human COPD lung tissue using IL-9 antibody.

FIG. 4: Immunohistochemistry analysis of paraffin sections of human COPD lung tissue using IL-9 antibody.

FIG. 5: Chronic challenge protocol.

FIG. 6a-6g: Chronic allergen challenge induces mucus production and sub-epithelial collagen deposition, which are attenuated by IL-9 blockade. (a) H&E (inflammation) staining of sham treated and OVA treated mice; (b) PAS (mucus) staining of sham treated and OVA treated mice; (c) H&E staining of OVA challenged mice that received IgG negative control antibody versus OVA challenged mice that received IL-9 antibody; (d) PAS staining of OVA challenged mice that received IgG negative control antibody versus OVA challenged mice that received IL-9 antibody; (e) total lung collagen and (f) peribronchial-associated collagen in sham and OVA treated mice. (g) representative Sirius Red sections for each group. Results are expressed as mean±sem. Significant differences between OVA wild type mice and OVA anti IL-9 antibody treated mice are shown as ***p<0.003-0.0001.

FIG. 7: IL-9 blockade significantly improves lung function following chronic allergen challenge. (a) resistance, (b) elastance, (c) Newtonian resistance, (d) tissue dampening, (e) tissue elastance, and (f) hysteresivity measures of lung function for chronic allergen challenged mice (), sham challenge control mice (∘), and chronic allergen challenged mice treated with anti IL-9 antibody (▪). Results are expressed as mean responses±sem.

FIG. 8: Anti IL-9 antibody significantly attenuates mast cell activation/numbers in tissue. Results are expressed as means±sem (sham group n=29-31; OVA mice n=34). Significant differences between the respective Sham and OVA Control IgG vs Anti IL-9 antibody groups are shown as ***p<0.0001.

FIG. 9: IL-9 blockade was associated with reduced levels of pro-fibrotic mediators in lung tissue following chronic OVA challenge. Effect of IL-9 antibody on levels of (a) TGF-β1, (b) VEGF, and (c) FGF-2 following OVA challenge. Results are expressed as means±sem (sham group n=10-12; OVA mice n=18-20). Significant differences between the respective sham and OVA control IgG vs anti IL-9 antibody groups are shown as *p<0.01 ***p<0.001-0001.

FIGS. 10a and 10b: Amino acid sequences of the (a) VH and (b) VL domain of antibody 4D4 (CDRs underlined).

FIGS. 11a and 11b: Amino acid sequences of the (a) VH and (b) VL domain of antibody 4D4H2-1 D11 (CDRs underlined).

FIGS. 12a and 12b: Amino acid sequences of the (a) VH and (b) VL domain of antibody 4D4com-XF-9 (CDRs underlined).

FIGS. 13a and 13b: Amino acid sequences of the (a) VH and (b) VL domain of antibody 4D4com-2F-9 (CDRs underlined).

FIGS. 14a and 14b: Amino acid sequences of the (a) VH and (b) VL domain of antibody 7F3 (CDRs underlined).

FIGS. 15a and 15b: Amino acid sequences of the (a) VH and (b) VL domain of antibody 71A10 (CDRs underlined).

FIGS. 16a and 16b: Amino acid sequences of the (a) VH and (b) VL domain of antibody 7F3 22D3 (CDRs underlined).

FIGS. 17a and 17b: Amino acid sequences of the (a) VH and (b) VL domain of antibody 7F3com-2H2 (CDRs underlined).

FIGS. 18a and 18b: Amino acid sequences of the (a) VH and (b) VL domain of antibody 7F3com-3H5 (CDRs underlined).

FIGS. 19a and 19b: Amino acid sequences of the (a) VH and (b) VL domain of antibody 7F3com-3D4 (CDRs underlined).

FIGS. 20a and 20(b): Nucleotide sequences encoding the (a) VH and (b) VL domain of antibody 7F3com-2H2 (CDRs underlined).

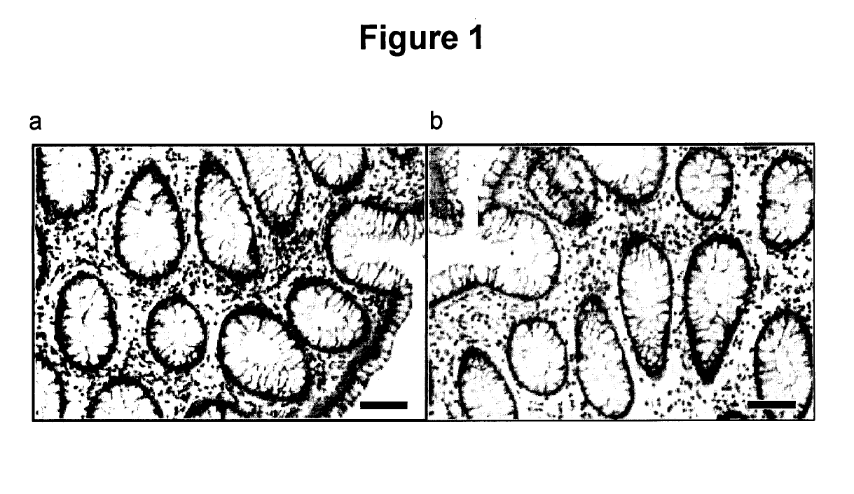

### DETAILED DESCRIPTION OF THE INVENTION

Disorders/Diseases

The invention encompasses treatment of disorders, e.g., fibrotic and inflammatory disorders, by administering inhibitors of IL-9. Fibrosis or fibrotic conditions may generally be described as conditions which develop as the result of formation of excess fibrous connective tissue in an organ or tissue of a subject as a reparative or reactive process. The inhibitors of IL-9 may be used to treat any such condition. Fibrotic conditions which may be treated by the inhibitors of IL-9 may be diseases/disorders that effect internal organs (e.g., liver, lung, kidney, heart blood vessels, gastrointestinal tract), and may occur in disorders such as pulmonary fibrosis, myelofibrosis, liver cirrhosis, mesangial proliferative glomerulonephritis, crescentic glomerulonephritis, diabetic nephropathy, renal interstitial fibrosis, renal fibrosis in patients receiving cyclosporin, and HIV associated nephropathy. Dermal fibrosing disorders include, but are not limited to, scleroderma, morphea, keloids, hypertrophic scars, familial cutaneous collagenoma, and connective tissue nevi of the collagen type. Fibrotic conditions of the eye include conditions such as diabetic retinopathy, postsurgical scarring (for example, after glaucoma filtering surgery and after cross-eye surgery), and proliferative vitreoretinopathy. Additional fibrotic conditions include: rheumatoid arthritis, diseases associated with prolonged joint pain and deteriorated joints; progressive systemic sclerosis, polymyositis, dermatomyositis, eosinophilic fascitis, morphea, Raynaud's syndrome, and nasal polyposis.

Inflammatory disorders include, but are not limited to, asthma, allergic disorders, inflammatory disorders characterized by type-2 mediated inflammation, pulmonary fibrosis, chronic obstructive pulmonary disease (COPD), encephilitis, inflammatory bowel disease (e.g., ulcerative colitis, Chrohn's disease, and celiac disease), septic shock, undifferentiated spondyloarthropathy, undifferentiated arthropathy, arthritis, inflammatory osteolysis, and chronic inflammation resulting from chronic viral or bacteria infections. The inflammatory disorder may be characterized as a type 2-mediated inflammation. Type 2-mediated inflammation is characterized by eosinophilic and basophilic tissue infiltration and/or extensive mast cell degranulation, a process dependent on cross-linking of surface-bound IgE.

Treatment of Diseases/Disorders

Treatment of diseases/disorders refers to the reduction or amelioration of the progression, severity, and/or duration of a disease or disorder (e.g., a fibrotic or inflammatory disease or disorder) or the amelioration of one or more symptoms thereof. Treatment may result in a reduction in the swelling of organs or tissues, or a reduction in the pain associated with a gastrointestinal or respiratory condition. Treatment may result in a reduction in the inflammation associated with COPD. Treatment may result in reduction of the release of inflammatory agents by mast cells, or the reduction of the biological effect of such inflammatory agents. Treatment may result in weight gain, increased energy, increased appetite, decreased joint pain, or decreased bleeding associated with ulcerative colitis.

Treatment may also be related to prevention of the development or onset of a disease or disorder (e.g., a fibrotic or inflammatory disease or disorder) or one or more symptoms thereof) or the prevention of the recurrence, onset, or development of one or more symptoms of such a disease or disorder.

Subjects

Subjects may be patients that have any of the described fibrotic or inflammatory diseases or disorders. Subjects may be animals, e.g., a mammal including a non-primate (e.g., a cow, pig, horse, cat, dog, rat, and mouse) or a primate (e.g., a monkey, such as a cynomolgous monkey, chimpanzee, and a human). The subject may be a mammal, e.g., a human, with a disease or disorder (e.g., a fibrotic or inflammatory disease or disorder) or one or more symptoms of such a disease or disorder. The subject may be a farm animal (e.g., a horse, pig, or cow) or a pet (e.g., a dog or cat) with a disorder (e.g., a fibrotic or inflammatory disease or disorder) or one or more symptoms thereof. The subject may be a mammal (e.g., an immunocompromised or immunosuppressed mammal), e.g., a human, at risk of developing a disorder (e.g., a fibrotic or inflammatory disease or disorder or one or more symptoms thereof). The subject may or may not be a mammal which is not immunocompromised or immunosuppressed. The subject may be a mammal, e.g., a human, with a lymphocyte count not under 500 cells/mm³. The subject may be a human infant, a human infant born prematurely, a human child, a human adult, or an elderly human. The subject may suffer from inflammatory bowel disease, e.g., ulcerative colitis, or may suffer from COPD. The subject may be administered an inhibitor of IL-9 as a first therapy for a disease or disorder. The subject may be administered an inhibitor of IL-9 as a second or subsequent therapy for a disease or disorder. The subject may be refractory to one or more therapies other than an inhibitor of IL-9. The subject may be one identified as having a predisposition for a disease or disorder.

### Inhibitors of IL-9

Fibrotic or inflammatory diseases or disorders are treated by administration of an inhibitor of IL-9. An inhibitor of IL-9 may also be referred to as an antagonist of IL-9. Inhibitors or antagonists of IL-9 may be any protein, polypeptide, peptide, peptidomimetic, glycoprotein, antibody, antibody fragment, carbohydrate, nucleic acid, organic molecule, inorganic molecule, large molecule, or small molecule that blocks, inhibits, reduces or neutralizes the function, activity and/or expression of IL-9. An IL-9 inhibitor may reduce the function, activity and/or expression of IL-9 by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

Small molecules that inhibit IL-9 include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such agents.

Inhibitors of IL-9 may be present in an isolated form or in an isolated form in a composition or pharmaceutical composition. Compositions that comprise an organic or inorganic molecule (whether it be a small or large molecule), other than a proteinaceous agent or nucleic acid molecule, that are in an isolated form may be substantially free of a different organic or inorganic molecule. For instance, an isolated organic or inorganic molecule may be 60%, 65%, 70%, 75%, 80%, 85%, 90%, 95%, or 99% free of a second, different organic or inorganic molecule. A composition comprising a proteinaceous agent (e.g., a peptide, polypeptide, fusion protein, or antibody) in isolated form refers to a proteinaceous agent which is substantially free of cellular material or contaminating proteins from the cell or tissue source from which it is derived, or substantially free of chemical precursors or other chemicals when chemically synthesized. A proteinaceous agent that is substantially free of cellular material may include preparations of a proteinaceous agent having less than about 30%, 20%, 10%, or 5% (by dry weight) of heterologous protein, polypeptide, peptide, or antibody (also referred to as a “contaminating protein”).

Antibodies Specific for IL-9

The inhibitors of IL-9 employed by the methods encompassed by invention may be antibodies specific or immunospecific for IL-9, or antibodies that immunospecifically bind to an IL-9 polypeptide (e.g., a human IL-9 polypeptide). Antibody and antibodies immunospecific for IL-9 include monoclonal antibodies, multispecific antibodies, human antibodies, humanized antibodies, camelised antibodies, chimeric antibodies, single-chain Fvs (scFv), single chain antibodies, single domain antibodies, Fab fragments, F(ab′) fragments, disulfide-linked Fvs (sdFv), and anti-idiotypic (anti-Id) antibodies (including, e.g., anti-Id antibodies to antibodies of the invention), intrabodies, and epitope-binding fragments of any of the above. Antibodies that immunospecifically bind IL-9 include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

The antibodies that immunospecifically bind IL-9 may bind to an IL-9 polypeptide expressed by an immune cell such as an activated T cell or a mast cell. The antibodies that immunospecifically bind IL-9 may modulate an activity or function of T cells, B cells, mast cells, neutrophils, and/or eosinophils. The antibodies that immunospecifically bind IL-9 may inhibit or reduce the infiltration of inflammatory cells into a tissue, joint, or organ of a subject and/or inhibit or reduce epithelial cell hyperplasia.

IL-9 or an IL-9 polypeptide may be an analog, derivative or a fragment of a mature and immature form of IL-9 (see, Van Snick et al., 1989, J. Exp. Med. 169:363-68 and Yang et al., 1989, Blood 74:1880-84). The IL-9 polypeptide may be from any species. The nucleotide and/or amino acid sequences of IL-9 polypeptides can be found in the literature or public databases, or the nucleotide and/or amino acid sequences can be determined using cloning and sequencing techniques known to one of skill in the art. For example, the nucleotide sequence of human IL-9 can be found in the GenBank database (see, e.g., Accession No. NM_{—}000590). The amino acid sequence of human IL-9 can be found in the GenBank database (see, e.g., Accession Nos. P15248, NP_{—}000584 and AAC17735) and in U.S. Provisional Application No. 60/371,683, entitled, “Recombinant Anti-Interleukin-9 Antibodies,” filed Apr. 12, 2002 (the amino acid sequence of human IL-9 on page 15 is specifically incorporated herein by reference).

The antibody that immunospecifically binds IL-9 may inhibit and/or reduce the interaction between the IL-9 polypeptide and the IL-9 receptor (“IL-9R”) or a subunit thereof by approximately 25%, approximately 30%, approximately 35%, approximately 45%, approximately 50%, approximately 55%, approximately 60%, approximately 65%, approximately 70%, approximately 75%, approximately 80%, approximately 85%, approximately 90%, approximately 95%, or approximately 98% relative to a control such as PBS or a control IgG antibody in an in vivo and/or in vitro assay described herein or well-known to one of skill in the art (e.g., an immunoassay such as an ELISA).

The antibodies that immunospecifically bind IL-9 may inhibit or reduce the interaction between the IL-9 polypeptide and the IL-9 receptor (“IL-9R”) or one or more subunits thereof by at least 25%, at least 30%, at least 35%, at least 45%, at least 50%, at least 55%, at least 60%, at least 65%, at least 70%, at least 75%, at least 80%, at least 85%, at least 90%, at least 95%, or at least 98% relative to a control such as phosphate buffered saline (“PBS”) or a control IgG antibody in an in vivo and/or in vitro assay described herein or well-known to one of skill in the art (e.g., a cell proliferation assay using an IL-9 dependent cell line such as an IL-9 dependent mouse T cell line expressing the human IL-9R). Alternatively, the antibodies that immunospecifically bind IL-9 do not inhibit the interaction between an IL-9 polypeptide and the IL-9R or one or more subunits thereof relative to a control such as PBS or a control IgG antibody in an in vivo and/or in vitro assay described herein or well-known to one of skill in the art (e.g., a cell proliferation assay using an IL-9 dependent cell line such as an IL-9 dependent mouse T cell line expressing the human IL-9R). Alternatively, the antibodies that immunospecifically bind IL-9 may inhibit the interaction between the IL-9 polypeptide and the IL-9R or one or more subunits thereof by less than 20%, less than 15%, less than 10%, or less than 5% relative to a control such as PBS or a control IgG antibody in vivo and/or in vitro assay described herein or well-known to one of skill in the art, (e.g., a cell proliferation assay using an IL-9 dependent cell line such as an IL-9 dependent mouse T cell line expressing the human IL-9R).

The IL-9 antibody that immunospecifically binds IL-9 may induce a decrease in the concentration of cytokines such as, e.g., IL-4, IL-5, IL-10, IL-13, and IL-23 in the serum of a subject administered such an antibody relative to the concentration of such cytokines in the serum of a subject administered a control such as PBS or a control IgG antibody. Alternatively, the antibody that immunospecifically binds IL-9 may induce a decrease in the concentration of cytokines produced by mast cells, such as TNF-α, IL-4, and IL-13, in the serum of a subject administered such an antibody relative to the concentration of such cytokines in the serum of a subject administered a control such as PBS or a control IgG antibody. The IL-9 antibody that immunospecifically binds IL-9 may induce a decrease in the concentration of cytokines produced by Th2 cells, such as IL-4, IL-5, IL-13, and IL-10, in the serum of a subject administered such an antibody relative to the concentration of such cytokines in the serum of a subject administered a control such as PBS or a control IgG antibody. Serum concentrations of a cytokine can be measured by any technique well-known to one of skill in the art such as, e.g., ELISA or Western blot assay.

The IL-9 antibody that immunospecifically binds IL-9 may reduce and/or inhibit proliferation of inflammatory cells (e.g., mast cells, T cells, B cells, macrophages, neutrophils, basophils, and/or eosinophils) by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art (e.g., a trypan blue assay or ³H-thymidine assay). The IL-9 antibody that immunospecifically binds IL-9 may reduce and/or inhibit infiltration of inflammatory cells into the upper and/or lower respiratory and/or gastrointestinal tract by at least at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art. The IL-9 antibody that immunospecifically binds IL-9 may both (a) reduce and/or inhibit infiltration of inflammatory cells into the upper respiratory and/or lower respiratory and/or gastrointestinal tract and (b) reduce and/or inhibit proliferation of the inflammatory cells by at least 25%, preferably at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well known in the art.

The IL-9 antibody that immunospecifically binds IL-9 may reduce mast cell degranulation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art (see, e.g., Windmiller and Backer, 2003, J. Biol. Chem. 278:11874-78 for examples of mast cell degranulation assays). The IL-9 antibody that immunospecifically binds IL-9 may inhibit and/or reduce mast cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art. The IL-9 antibody that immunospecifically binds IL-9 may inhibit and/or reduce the expression and/or release of products of mast cell activation and/or degranulation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described or well-known to one of skill in the art.

The IL-9 antibody that immunospecifically binds IL-9 may inhibit and/or reduce the expression, activity, serum concentration, and/or release of mast cell proteases, such as chymase and tryptase, by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well known to one of skill in the art. The mast cell activity may be measured by culturing primary mast cells or a mast cell line in vitro in the presence of 10 ng/ml of IL-9. Baseline levels of protease (e.g., chymase and tryptase) and leukotriene are determined in the supernatant by commercially available ELISA kits. The ability of antibodies to modulate protease or leukotriene levels is assessed by adding an IL-9-reactive antibody or control antibody directly to cell cultures at a concentration of 1 μg/ml. Protease and leukotriene levels are assessed at 24 and 36 hour timepoints. The IL-9 antibody that immunospecifically binds IL-9 may inhibit and/or reduce the expression, activity, serum concentration, and/or release of mast cell leukotrienes, such as C4, D4, and E4 by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art. The IL-9 antibody that immunospecifically binds IL-9 may inhibit and/or reduce the expression, activity, serum concentration, and/or release of mast cell cytokines, such as TNF-α, IL-4, and IL-13 by at least 25%, preferably at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art (e.g., an ELISA or Western blot assay).

The IL-9 antibody that immunospecifically binds IL-9 may inhibit and/or reduce mast cell infiltration by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known in the art. The IL-9 antibody that immunospecifically binds IL-9 may inhibit and/or reduce mast cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art (e.g., a trypan blue assay, FACS or ³H thymidine assay). The IL-9 antibody that immunospecifically binds IL-9 may inhibit and/or reduce mast cell infiltration by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vitro and/or in vivo assay described herein or well-known in the art and inhibit and/or reduce mast cell proliferation at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art (e.g., a trypan blue assay, FACS or ³H thymidine assay). Reductions in mast cell infiltration may be measured in vivo by sensitizing animals to ovalbumin. Briefly, 100 μg of ovalbumin complexed with aluminum adjuvant is administered subcutaneously on days 1 and 21. Throughout the three-week sensitization procedure, animals are administered an IL-9 reactive antibody or a control antibody at a 10 mg/kg dose every 5 to 7 days. On days 29, 30 and 31, animals are exposed to ovalbumin without adjuvant by aerosol delivery, or alternatively, by intranasal instillation of 100 μl of a 1 μg/ml solution prepared in PBS. On day 31, 6 hours after the last ovalbumin challenge, animals are euthanized and lung tissue is fixed by perfusion with formalin. Mast cell infiltration is assessed histologically by counting mast cells per field in lung epithelial tissue sections. Using this experimental design, mast cell precursors may be differentiated from mast cells in lung epithelium by assessing (for example) whether metachromatic granules are present, and/or by immunohistochemistry using differentiation-dependent cell surface markers (e.g., FcepsilonRI).

The IL-9 antibody that immunospecifically binds IL-9 may reduce eosinophil, B cell, T cell or neutrophil infiltration in the upper respiratory and/or lower respiratory and/or gastrointestinal tracts by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well known to one of skill in the art (see, e.g., Li et al., 2000, Am. J. Respir. Cell Mol. Biol. 25:644-51). The IL-9 antibody that immunospecifically binds IL-9 may inhibit and/or reduce eosinophil, B cell, T cell, or neutrophil proliferation, by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay (e.g., a trypan blue assay, FACS or ³H thymidine assay). The IL-9 antibody that immunospecifically binds IL-9 may inhibit and/or reduce eosinophil, B cell, T cell, or neutrophil infiltration by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art and inhibits and/or reduces eosinophil, T cell, B cell, or neutrophil proliferation at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well known to one of skill in the art.

The IL-9 antibody that immunospecifically binds IL-9 may neutralize or inhibit IL-9 mediated biological effects including, but not limited to inflammatory cell recruitment, epithelia hyperplasia, mucin production of epithelial cells, and mast cell activation, degranulation, proliferation, and/or infiltration.

The IL-9 antibody that immunospecifically binds IL-9 may act synergistically with a proteinaceous agent (e.g., a peptide, polypeptide, or protein (including an antibody)) and/or a non-proteinaceous agent that antagonizes the expression, function, and/or activity of IgE to reduce or inhibit the activation, degranulation, proliferation, and/or infiltration of mast cells by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assays described herein or well known to one of skill in the art.

The IL-9 antibody that immunospecifically binds IL-9 may act synergistically with a proteinaceous agent (e.g., a peptide, polypeptide, protein (including an antibody)) and/or a non-proteinaceous agent that antagonizes the expression, function, and/or activity of a mast cell protease to reduce or inhibit the activation, degranulation, proliferation, and/or infiltration of mast cells by at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art.

The IL-9 antibody that immunospecifically binds IL-9 may act synergistically with a proteinaceous agent (e.g., a peptide, polypeptide, and protein (including an antibody)) or a non-proteinaceous agent that antagonizes the expression, function, and/or activity of a stem cell factor to reduce or inhibit to reduce or inhibit the activation, degranulation, proliferation, and/or infiltration of mast cells by at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art. Primary mast cells or a mast cell line may be cultured in vitro in the presence of 1 ng/ml IL-9 plus 1 ng/ml stem cell factor. Baseline levels of protease (e.g., chymase and tryptase) and leukotriene are determined in the supernatant by commercially available ELISA kits. The ability of antibodies to modulate protease or leukotriene levels can be assessed by adding IL-9 reactive antibody or control antibody directly to cell cultures at a concentration of 1 μg/ml. Protease and leukotriene levels can be assessed at 24 and 36 hour time points.

The IL-9 antibody that immunospecifically binds IL-9 may be monospecific, bispecific, trispecific or of greater multispecificity. Multispecific antibodies may be specific for different epitopes of an IL-9 polypeptide or may be specific for both an IL-9 polypeptide as well as for a heterologous epitope, such as a heterologous polypeptide or solid support material. See, e.g., International publications WO 93/17715, WO 92/08802, WO 91/00360, and WO 92/05793; Tutt, et al., J. Immunol. 147:60-69(1991); U.S. Pat. Nos. 4,474,893, 4,714,681, 4,925,648, 5,573,920, and 5,601,819; and Kostelny et al., J. Immunol. 148:1547-1553 (1992).

The IL-9 antibody that immunospecifically binds IL-9 may have high binding affinity for an IL-9 polypeptide. The IL-9 antibody that immunospecifically binds IL-9 may have an association rate constant or kₒₙ rate (antibody (Ab)+antigen (Ag)(kₒₙ→Ab-Ag) of at least 10⁵ M⁻¹s⁻¹, at least 1.5×10⁵ M⁻¹s⁻¹, at least 2×10⁵ M⁻¹s⁻¹, at least 2.5×10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹, or 10⁵-10⁸ M⁻¹s⁻¹, 1.5×10⁵ M⁻¹s⁻¹-1×10⁷ M⁻¹s⁻, 2×10⁵-1×10⁶ M⁻¹s⁻¹, or 4.5×10^{5×107} M⁻¹s⁻¹. The IL-9 antibody that immunospecifically binds IL-9 may have a kₒₙ of at least 2×10⁵ M⁻¹s⁻¹, at least 2.5×10⁵ M⁻¹s⁻¹, at least 5×10⁵ M⁻¹s⁻¹, at least 10⁶M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹ as determined by a BIAcore assay and may further neutralize human IL-9 in the microneutralization assay. The IL-9 antibody that immunospecifically binds IL-9 may have a kₒₙ of at most 10⁸ M⁻¹s⁻¹, at most 10⁹ M⁻¹s⁻¹, at most 10¹⁰ M⁻¹s⁻¹, at most 10¹¹ M⁻¹s⁻¹, or at most 10¹² M⁻¹s⁻¹ as determined by a BIAcore assay and may further neutralize human IL-9.

The IL-9 antibody that immunospecifically binds IL-9 may have a k_{off} rate (antibody (Ab)+antigen (Ag k_{off}⇄Ab-Ag) of less than 10⁻³ s⁻¹, less than 5×10⁻³ s⁻¹, less than 10⁻⁴ s⁻¹, less than 2×10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹, or 10⁻³-10⁻¹⁰ s⁻¹, 10⁻⁴-10⁻⁸ s⁻¹, or 10⁻⁵-10⁻⁸ s⁻¹. The IL-9 antibody that immunospecifically binds IL-9 may have a k_{off} of 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹ or less than 10⁻¹⁰ s⁻¹ as determined by a BIAcore assay and may further neutralize human IL-9 in microneutralization assay. The IL-9 antibody that immunospecifically binds IL-9 may have a k_{off} of greater than 10⁻¹³ s⁻¹, greater than 10⁻¹² s⁻¹, greater than 10⁻¹¹ s⁻¹, greater than 10⁻¹⁰ s⁻¹, greater than 10⁻⁹ s⁻¹, or greater than 10⁻⁸ s⁻¹.

The IL-9 antibody that immunospecifically binds IL-9 may have an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10^{11 M}⁻¹, at least 5×10¹¹ M⁻¹, at least 10¹² M⁻¹, at least 5×10¹² M⁻¹, at least 10^{—}M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹, or 10²-5×10⁵ M⁻¹, 10⁴-1×10¹⁰ M⁻¹, or 10⁵-1×10⁸ M⁻¹. The IL-9 antibody that immunospecifically binds IL-9 may have a Kₐ of at most 10¹¹ M⁻¹, at most 5×10¹¹ M⁻¹, at most 10¹² M⁻¹, at most 5×10¹² M⁻¹, at most 10¹³ M⁻¹, at most 5×10¹³ M⁻¹, at most 10¹⁴ M⁻¹, or at most 5×10¹⁴ M⁻¹. The IL-9 antibody that immunospecifically binds IL-9 may have a dissociation constant or K_{d} (kₒₙ/k_{off}) of less than 10⁻⁵ M, less than 5×10⁻⁵ M, less than 10⁻⁶ M, less than 5×10⁻⁶ M, less than 10⁻⁷ M, less than 5×10⁻⁷, less than 10⁻⁸ M, less than 4×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴M, less than 10⁻¹⁵ M, or less than 5×10⁻¹⁵ M or 10⁻² M-5×10⁻⁵ M, 10⁻⁶-10⁻¹⁵ M, or 10⁻⁸-10⁻¹⁴ M. The IL-9 antibody that immunospecifically binds IL-9 may have a K_{d} of less than 10⁻⁹ M, less than 5×10⁻⁹ M, less than 10⁻¹⁰ M, less than 5×10⁻¹⁰ M, less than 1×10⁻¹¹ M, less than 5×10⁻¹¹ M, less than 1×10⁻¹² M, less than 5×10⁻¹² M, less than 10⁻¹³ M, less than 5×10⁻¹³ M or less than 1×10⁻¹⁴ M, or 10⁻⁹ M-10⁻¹⁴ M as determined by a BIAcore assay and the antibody may further human IL-9 in a microneutralization assay. The IL-9 antibody that immunospecifically binds IL-9 may have a K_{d} of greater than 10⁻⁹ M, greater than 5×10⁻⁹ M, greater than 10⁻¹⁰ M, greater than 5×10⁻¹⁰ M, greater than 10⁻¹¹ M, greater than 5×10⁻¹¹ M, greater than 10⁻¹² M, greater than 5×10⁻¹² M, greater than 6×10⁻¹² M, greater than 10⁻¹³ M, greater than 5×10⁻¹³ M, greater than 10⁻¹⁴ M, greater than 5×10⁻¹⁴ M or greater than 10⁻⁹ M-10⁻¹⁴ M.

Antibodies that immunospecifically bind IL-9 may have an extended half-life in vivo. The antibodies that immunospecifically bind IL-9 may have a half life of greater than 3 days, greater than 7 days, greater than 10 days, greater than 15 days, greater than 25 days, greater than 30 days, greater than 35 days, greater than 40 days, greater than 45 days, greater than 2 months, greater than 3 months, greater than 4 months, or greater than 5 months in a subject, e.g., a mammal. Half-life serum circulation of antibodies (e.g., monoclonal antibodies, single chain antibodies and Fab fragments) in vivo, may be increased, for example, by attaching inert polymer molecules such as high molecular weight polyethyleneglycol (PEG) to the antibodies with or without a multifunctional linker either through site-specific conjugation of the PEG to the N- or C-terminus of the antibodies or via epsilon-amino groups present on lysine residues. Linear or branched polymer derivatization that results in minimal loss of biological activity can be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art.

Antibodies having an increased half-life in vivo can also be generated introducing one or more amino acid modifications (i.e., substitutions, insertions or deletions) into an IgG constant domain, or FcRn binding fragment thereof (preferably a Fc or hinge-Fc domain fragment). See, e.g., International Publication No. WO 98/23289; International Publication No. WO 97/34631; International Publication No. WO 02/060919; and U.S. Pat. No. 6,277,375, each of which is incorporated herein by reference in its entirety.

Further, antibodies can be conjugated to albumin in order to make the antibody or antibody fragment more stable in vivo or have a longer half life in vivo. The techniques are well-known in the art, see, e.g., International Publication Nos. WO 93/15199, WO 93/15200, and WO 01/77137; and European Patent No. EP 413,622.

Antibodies that immunospecifically bind IL-9 may be recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. The fusion proteins may comprise an antigen-binding fragment of an IL-9 immunospecific antibody (e.g., a Fab fragment, Fd fragment, Fv fragment, F(ab)₂ fragment, a VH domain, a VH CDR, a VL domain or a VL CDR) and a heterologous protein, polypeptide, or peptide. The heterologous protein, polypeptide, or peptide that the antibody or antibody fragment is fused to may useful for targeting the antibody to respiratory epithelial cells, mast cells, neutrophils, eosinophils, B cells, macrophages, or activated T cells. For example, an antibody that immunospecifically binds to a cell surface receptor expressed by a particular cell type (e.g., a respiratory epithelial cell, a mast cell, a neutrophil, an eosinophil, a B cell, a macrophage, or an activated T cell) may be fused or conjugated to an antibody or fragment immunospecific for IL-9. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313. Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that immunospecifically binds to an IL-9 polypeptide may be recombined with one or more components, motifs, sections, parts, domains, fragments, etc. of one or more heterologous molecules.

Moreover, the antibodies or fragments thereof can be fused to marker sequences, such as a peptide to facilitate purification. For example, the marker amino acid sequence may be a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

Antibodies immunospecific for IL-9 may be conjugated to a diagnostic or detectable agent. Such agents include, but are not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (131I, ¹²⁵I, ¹²³I, and ¹²¹I,), carbon (¹⁴C), sulfur (³⁵S), tritium (³H), indium (¹¹⁵In, ¹¹³In, ¹¹²In, and ¹¹¹In,), technetium (⁹⁹Tc), thallium (²⁰¹Ti), gallium (⁶⁸Ga, ⁶⁷Ga), palladium (¹⁰³Pd), molybdenum ⁹⁹Mo), xenon (¹³³Xe) fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Sn; and positron emitting metals using various positron emission tomographies, and noradioactive paramagnetic metal ions.

The antibody immunospecific for IL-9 may be conjugated to a therapeutic moiety. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Therapeutic moieties include, but are not limited to, antimetabolites (e.g., methotrexate, 6-mercaptopurine, 6-thioguanine, cytarabine, 5-fluorouracil decarbazine); alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum(II) (DDP), and cisplatin); anthracyclines (e.g., daunorubicin (formerly daunomycin) and doxorubicin); antibiotics (e.g., dactinomycin (formerly actinomycin), bleomycin, mithramycin, and anthramycin (AMC)); Auristatin molecules (e.g., auristatin PHE, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference); hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)); cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucorticoids, procaine, tetracaine, lidocaine, propranolol, and puromycin and analogs or homologs thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459); farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305); topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN-1518B; KT6006; KT6528; ED-110; NB-506; ED-110; NB-506; and rebeccamycin); bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate) HMG-CoA reductase inhibitors, (e.g., lovastatin, simvastatin, atorvastatin, pravastatin, fluvastatin, statin, cerivastatin, lescol, lupitor, rosuvastatin and atorvastatin); antisense oligonucleotides (e.g., those disclosed in the U.S. Pat. Nos. 6,277,832, 5,998,596, 5,885,834, 5,734,033, and 5,618,709); adenosine deaminase inhibitors (e.g., Fludarabine phosphate and 2-Chlorodeoxyadenosine); ibritumomab tiuxetan (Zevalin®); tositumomab (Bexxar®)) and pharmaceutically acceptable salts, solvates, clathrates, and prodrugs thereof.

Further, an antibody immunospecific for IL-9 may be conjugated to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567-1574), and VEGF (see, International Publication No. WO 99/23105), an anti-angiogenic agent, e.g., angiostatin, endostatin or a component of the coagulation pathway (e.g., tissue factor); or, a biological response modifier such as, for example, a lymphokine (e.g., interferon gamma (“IFN-γ”), interleukin-1 (“IL-1”), interleukin-2 (“IL-2”), interleukin-5 (“IL-5”), interleukin-6 (“IL-6”), interleukin-7 (“IL-7”), interleukin-10 (“IL-10”), interleukin-12 (“IL-12”), interleukin-15 (“IL-15”), interleukin-23 (“IL-23”), granulocyte macrophage colony stimulating factor (“GM-CSF”), and granulocyte colony stimulating factor (“G-CSF”)), or a growth factor (e.g., growth hormone (“GH”)), or a coagulation agent (e.g., calcium, vitamin K, tissue factors, such as but not limited to, Hageman factor (factor XII), high-molecular-weight kininogen (HMWK), prekallikrein (PK), coagulation proteins-factors II (prothrombin), factor V, XIIa, VIII, XIIIa, XI, XIa, IX, IXa, X, phospholipid fibrinopeptides A and B from the α and β chains of fibrinogen, fibrin monomer). In a specific embodiment, an antibody that immunospecifically binds to an IL-9 polypeptide is conjugated with a leukotriene antagonist (e.g., montelukast, zafirlukast, pranlukast, and zyleuton).

Moreover, an antibody immunospecific for IL-9 can be conjugated to therapeutic moieties such as a radioactive metal ion, such as alph-emiters such as ²¹³Bi or macrocyclic chelators useful for conjugating radiometal ions, including but not limited to, ¹³¹In, ¹³¹L, ¹³¹Y, ¹³¹Ho, ¹³¹Sm, topolypeptides or any of those listed supra. In certain embodiments, the macrocyclic chelator is 1,4,7,10-tetraazacyclodo- decane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Alternatively, an antibody immunospecific for IL-9 can be conjugated to a second antibody to form an antibody heteroconjugate as described by Segal in U.S. Pat. No. 4,676,980.

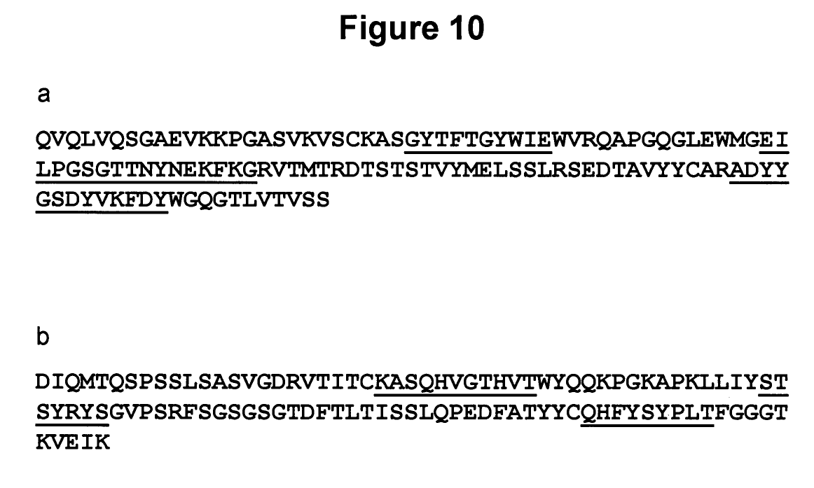

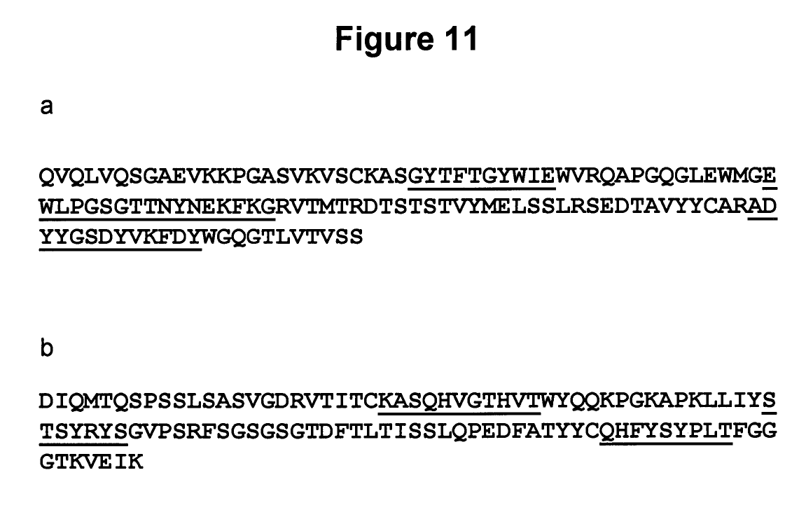

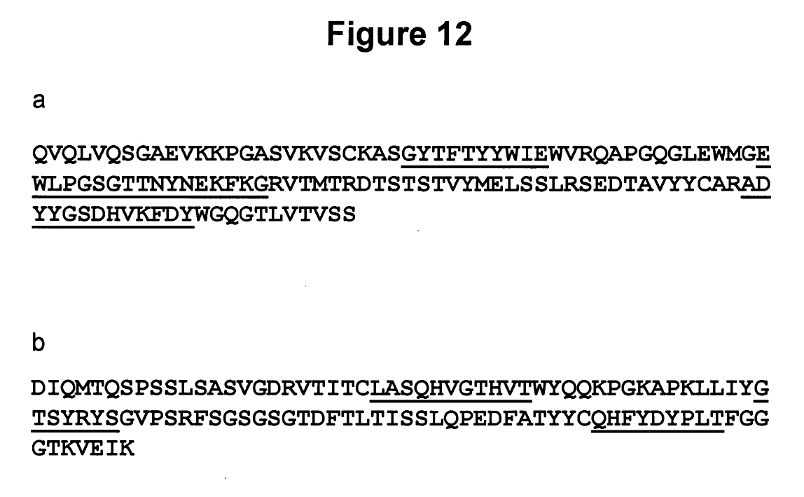

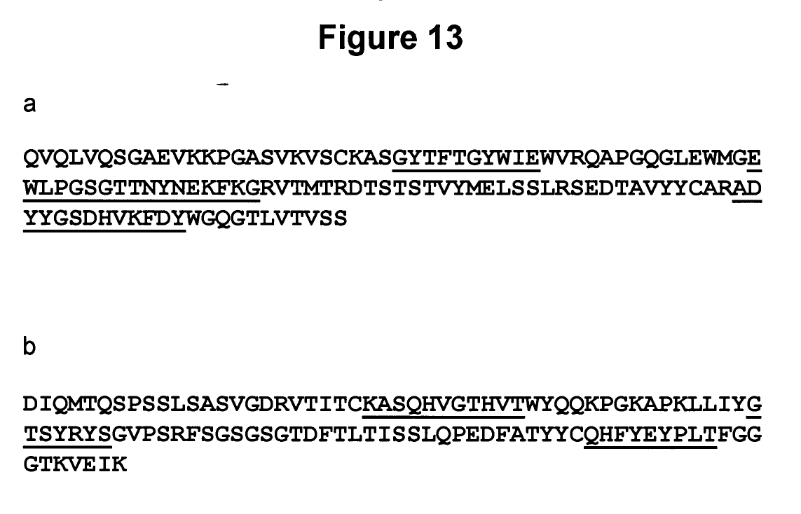

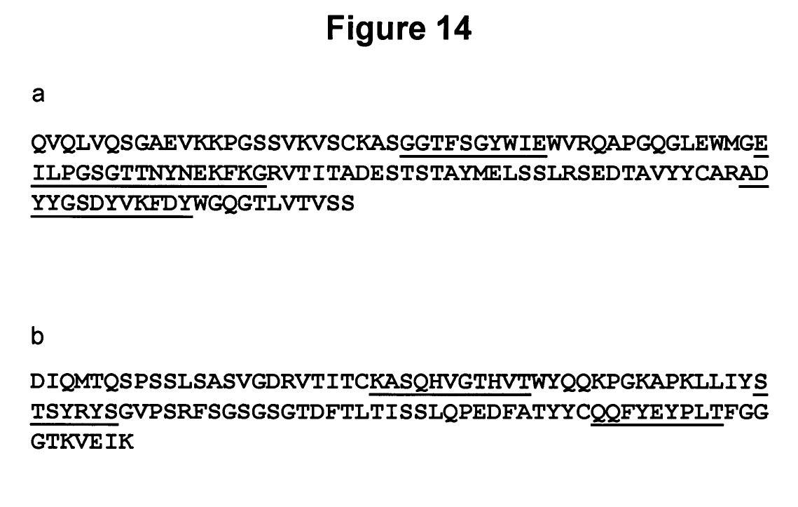

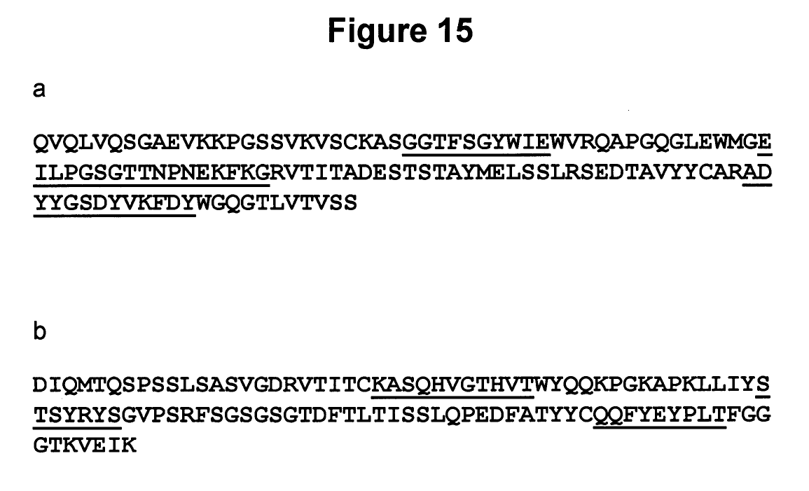

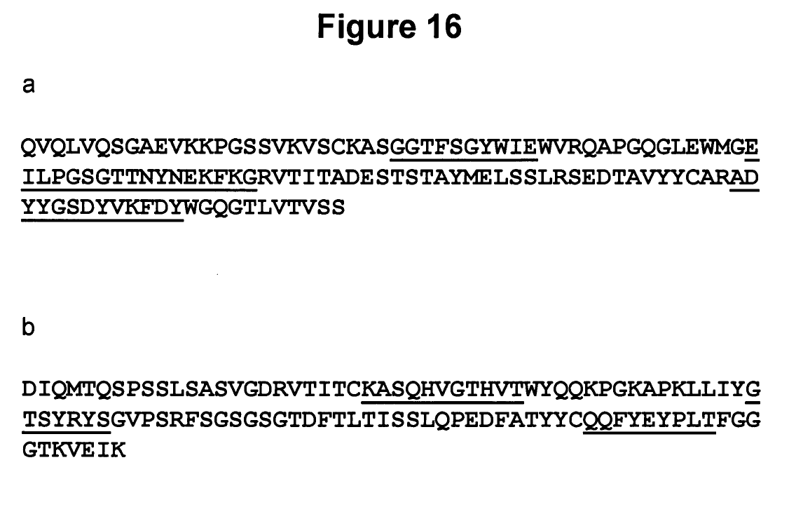

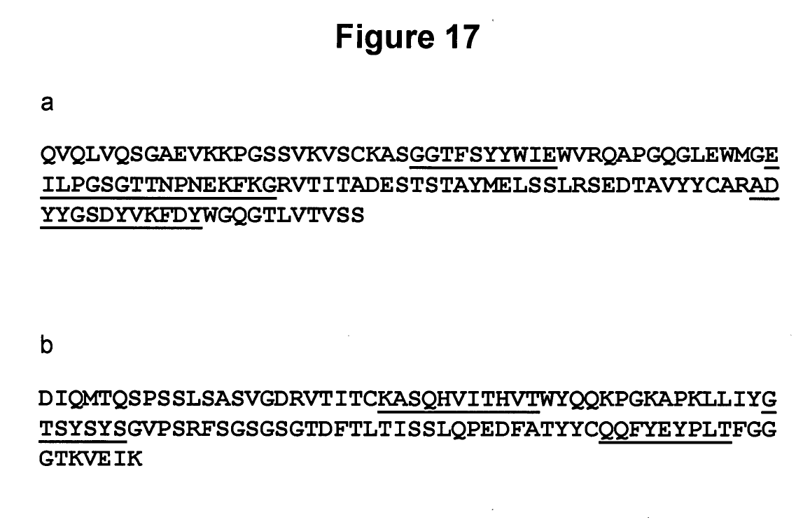

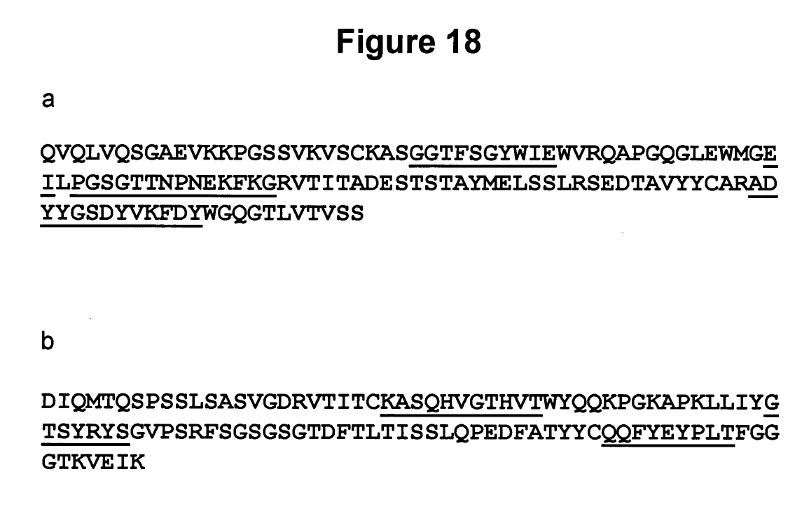

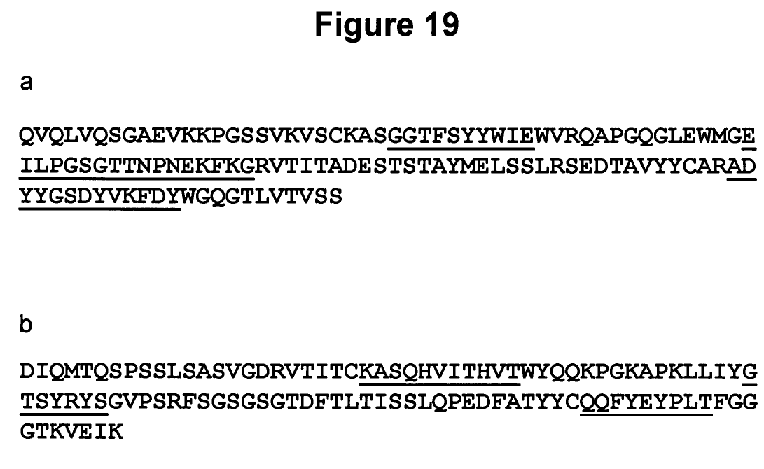

The following antibodies are provided as examples of antibodies specific for IL-9: 4D4, 4D4H2-1 D11, 4D4com-XF-9, 4D4com-2F9, 7F3, 71A10, 7F3 22D3, 7F3com-2H2, 7F3com-3H5, and 7F3com-3D4 disclosed in U.S. patent application publication 2005-0002934. FIGS. 10-19 provide the amino acid sequence of the variable light and heavy chains of each of these antibodies. The constant regions for each of the antibodies are identical to the constant regions of palivizumab (MedImmune, Inc.) IgG₁ (see U.S. Pat. No. 5,824,307, issued Oct. 20, 1998). Other non-limiting examples of antibodies specific for IL-9 have been disclosed in U.S. patent application publication 2003-0219439 and U.S. Pat. No. 6,261,559.

The antibodies that specifically bind IL-9 may comprise a VH domain having an amino acid sequence of the VH domain of 4D4, 4D4H2-1 D11, 4D4com-XF-9, 4D4com-2F9, 7F3, 71A10, 7F3 22D3, 7F3com-2H2, 7F3com-3H5, or 7F3com-3D4. The antibody that immunospecifically binds IL-9 may comprise a VH CDR1 having the amino acid sequence of the VH CDR1 of any one of antibodies 4D4, 4D4H2-1 D11, 4D4com-XF-9, 4D4com-2F9, 7F3, 71A10, 7F3 22D3, 7F3com-2H2, 7F3com-3H5, or 7F3com-3D4. The antibody that immunospecifically binds IL-9 may comprise a VH CDR2 having the amino acid sequence of VH CDR2 of any one of 4D4, 4D4H2-1 D11, 4D4com-XF-9, 4D4com-2F9, 7F3, 71A10, 7F3 22D3, 7F3com-2H2, 7F3com-3H5, or 7F3com-3D4. The antibody that immunospecifically binds IL-9 may comprise a VH CDR3 having the amino acid sequence of VH CDR3 of any one of 4D4, 4D4H2-1 D11, 4D4com-XF-9, 4D4com-2F9, 7F3, 71A10, 7F3 22D3, 7F3com-2H2, 7F3com-3H5, or 7F3com-3D4. The antibody that immunospecifically binds IL-9 may comprise a VH CDR1 and a VH CDR2 having the amino acid sequences of VH CDR1 and VH CDR2 of any one of the antibodies of 4D4, 4D4H2-1 D11, 4D4com-XF-9, 4D4com-2F9, 7F3, 71A10, 7F3 22D3, 7F3com-2H2, 7F3com-3H5, or 7F3com-3D4. The antibody that immunospecifically binds IL-9 polypeptide may comprise a VH CDR1 and a VH CDR3 having the amino acid sequence of VH CDR1 and VH CDR3 of any one of the antibodies of 4D4, 4D4H2-1 D11, 4D4com-XF-9, 4D4com-2F9, 7F3, 71A10, 7F3 22D3, 7F3com-2H2, 7F3com-3H5, or 7F3com-3D4. The antibody that immunospecifically binds to IL-9 may comprise a VH CDR2 and a VH CDR3 having the amino acid sequence of VH CDR2 and VH CDR3 of any one of the antibodies of 4D4, 4D4H2-1 D11, 4D4com-XF-9, 4D4com-2F9, 7F3, 71A10, 7F3 22D3, 7F3com-2H2, 7F3com-3H5, or 7F3com-3D4. The antibody that immunospecifically binds IL-9 may comprise a VH CDR1, a VH CDR2, and a VH CDR3 having the amino acid sequence of VH CDR1, VH CDR2 and VH CDR3 of any one of antibodies 4D4, 4D4H2-1 D11, 4D4com-XF-9, 4D4com-2F9, 7F3, 71A10, 7F3 22D3, 7F3com-2H2, 7F3com-3H5, or 7F3com-3D4.

The antibodies that immunospecifically bind IL-9, may comprise one, two, three, four, five or more VH CDRs. The VH CDRS may have an amino acid sequence of any of the VH CDRs of 4D4, 4D4H2-1 D11, 4D4com-XF-9, 4D4com-2F9, 7F3, 71A10, 7F3 22D3, 7F3com-2H2, 7F3com-3H5, or 7F3com-3D4.

The antibodies that immunospecifically bind IL-9 may comprise a VL domain having an amino acid sequence of the VL domain of any of antibodies 4D4, 4D4H2-1 D11, 4D4com-XF-9, 4D4com-2F9, 7F3, 71A10, 7F3 22D3, 7F3com-2H2, 7F3com-3H5, or 7F3com-3D4. The antibody that immunospecifically binds IL-9 may comprise a VL domain having the amino acid sequence of the VL domain of 7F3com-2H2. The antibody that immunospecifically binds IL-9 may comprise a VL CDR1 having the amino acid sequence of the VL CDR1 of any one of antibodies 4D4, 4D4H2-1 D11, 4D4com-XF-9, 4D4com-2F9, 7F3, 71A10, 7F3 22D3, 7F3com-2H2, 7F3com-3H5, or 7F3com-3D4. The antibody that immunospecifically binds IL-9 may comprise a VL CDR2 having the amino acid sequence of VL CDR2 of any one of 4D4, 4D4H2-1 D11, 4D4com-XF-9, 4D4com-2F9, 7F3, 71A10, 7F3 22D3, 7F3com-2H2, 7F3com-3H5, or 7F3com-3D4. The antibody that immunospecifically binds IL-9 may comprise a VL CDR3 having the amino acid sequence of VL CDR3 of any one of 4D4, 4D4H2-1 D11, 4D4com-XF-9, 4D4com-2F9, 7F3, 71A10, 7F3 22D3, 7F3com-2H2, 7F3com-3H5, or 7F3com-3D4. The antibody that immunospecifically binds IL-9 may comprise a VL CDR1 and a VL CDR2 having amino acid sequences of VL CDR1 and VL CDR2 of any one of the antibodies of 4D4, 4D4H2-1 D11, 4D4com-XF-9, 4D4com-2F9, 7F3, 71A10, 7F3 22D3, 7F3com-2H2, 7F3com-3H5, or 7F3com-3D4. The antibody that immunospecifically binds IL-9 polypeptide may comprise a VL CDR1 and a VL CDR3 having the amino acid sequence of VL CDR1 and VL CDR3 of any one of the antibodies of 4D4, 4D4H2-1 D11, 4D4com-XF-9, 4D4com-2F9, 7F3, 71A10, 7F3 22D3, 7F3com- 2H2, 7F3com-3H5, or 7F3com-3D4. The antibody that immunospecifically binds to IL-9 may comprise a VL CDR2 and a VL CDR3 having the amino acid sequence of VL CDR2 and VL CDR3 of any one of the antibodies of 4D4, 4D4H2-1 D11, 4D4com-XF-9, 4D4com-2F9, 7F3, 71A10, 7F3 22D3, 7F3com-2H2, 7F3com-3H5, or 7F3com-3D4. The antibody that immunospecifically binds IL-9 may comprise a VL CDR1, a VL CDR2, and a VL CDR3 having the amino acid sequence of VL CDR1, VL CDR2 and VL CDR3 of any one of antibodies 4D4, 4D4H2-1 D11, 4D4com-XF-9, 4D4com-2F9, 7F3, 71A10, 7F3 22D3, 7F3com-2H2, 7F3com-3H5, or 7F3com-3D4.

The antibodies that immunospecifically bind IL-9, may comprise one, two, three, four, five or more VL CDRs. The VL CDRS may have an amino acid sequence of any of the VL CDRs of 4D4, 4D4H2-1 D11, 4D4com-XF-9, 4D4com-2F9, 7F3, 71A10, 7F3 22D3, 7F3com-2H2, 7F3com-3H5, or 7F3com-3D4.

The antibodies that immunospecifically bind IL-9 may comprise a VH domain disclosed herein combined with a VL domain disclosed herein, or other VL domain (e.g., a VL domain disclosed in U.S. patent application publication 2003-0219439). The antibodies that immunospecifically bind IL-9 may comprise a VL domain disclosed herein combined with a VH domain disclosed herein, or other VH domain (e.g., a VL domain disclosed in U.S. patent application publication 2003-0219439).

The antibodies that immunospecifically bind IL-9 may comprise a VL CDR of one of 4D4, 4D4H2-1 D11, 4D4com-XF-9, 4D4com-2F9, 7F3, 71A10, 7F3 22D3, 7F3com-2H2, 7F3com-3H5, or 7F3com-3D4 and a VH CDR disclosed in U.S. patent application publication 2003-0219439. The antibodies that immunospecifically bind IL-9 polypeptide may comprise combinations of VH CDRs and VL CDRs described herein and disclosed in U.S. patent application publication 2003-02194392.

The antibodies that immunospecifically bind IL-9 may comprise one or more VH CDRs and one or more VL CDRs of any combination of 4D4, 4D4H2-1 D11, 4D4com-XF-9, 4D4com-2F9, 7F3, 71A10, 7F3 22D3, 7F3com-2H2, 7F3com-3H5, or 7F3com-3D4. The antibody that immunospecifically binds IL-9 may comprise a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs of 4D4, 4D4H2-1 D11, 4D4com-XF-9, 4D4com-2F9, 7F3, 71A10, 7F3 22D3, 7F3com-2H2, 7F3com-3H5, or 7F3com-3D4.

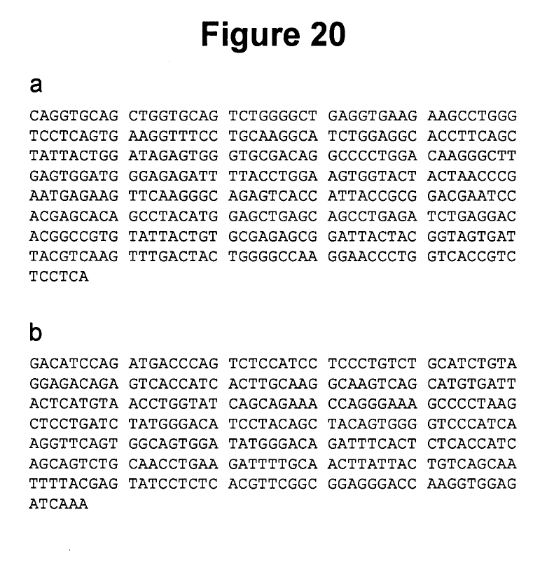

The antibody that immunospecifically binds IL-9 may comprise a VH CDR encoded by a nucleic acid sequence having a nucleotide sequence of a VH CDR of 7F3com-2H2 (FIG. 20). The antibody that immunospecifically binds IL-9 may comprise a VL CDR encoded by a nucleic acid sequence having a nucleotide sequence of a VL CDR of 7F3com-2H2 (FIG. 20). The antibody that immunospecifically binds IL-9 may comprise a VH CDR and a VL CDR encoded by a nucleic acid sequence having a nucleotide sequence of a VH CDR and a VL CDR of 7F3com-2H2 (FIG. 20).

The antibodies that immunospecifically bind IL-9 may comprise derivatives of the VH domains, VH CDRs, VL domains, or VL CDRs described herein. Standard techniques known to those of skill in the art can be used to introduce mutations (e.g., deletions, additions, and/or substitutions) in the nucleotide sequence encoding an antibody known to specifically bind IL-9, for example, site-directed mutagenesis and PCR-mediated mutagenesis which results in amino acid substitutions. The derivatives may include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. The derivatives may have conservative amino acid substitutions at one or more predicted non-essential amino acid residues (i.e., amino acid residues which are not critical for the antibody to immunospecifically bind to an IL-9 polypeptide). A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded antibody can be expressed and the activity of the antibody can be determined. The substitutions may be in the antibody CDRs, frameworks, or any other residue in variable or constant domains.

The antibodies that immunospecifically bind IL-9 may comprise the amino acid sequence of 4D4, 4D4 H2-1 D11, 4D4com-XF-9, 4D4com-2F9, 7F3, 71A10, 7F3 22D3, 7F3com-2H2, 7F3com-3H5, or 7F3com-3D4 with one or more amino acid residue substitutions in the variable light (VL) domain and/or variable heavy (VH) domain. The antibodies that immunospecifically bind IL-9 may comprise the amino acid sequence of 4D4, 4D4H2-1 D11, 4D4com-XF-9, 4D4com-2F9, 7F3, 71A10, 7F3 22D3, 7F3com-2H2, 7F3com-3H5, or 7F3com-3D4 with one or more amino acid residue substitutions in one or more VL CDRs (VL CDR1, VL CDR2, VL CDR3, VL CDR1 and CDR2, VL CDR1 and CDR3, VL CDR2 and CDR3, or VL CDR1, CDR2, and CDR3) and/or one or more VH CDRs (VH CDR1, VH CDR2, VH CDR3, VH CDR1 and CDR2, VH CDR1 and CDR3, VH CDR2 and CDR3, or VH CDR1, CDR2, and CDR3). The antibodies that immunospecifically bind IL-9 may comprise the amino acid sequence of 4D4, 4D4H2-1 D11, 4D4com-XF-9, 4D4com-2F9, 7F3, 71A10, 7F3 22D3, 7F3com-2H2, 7F3com-3H5, or 7F3com-3D4, or a VH and/or VL domain thereof with one or more amino acid residue substitutions in one or more VH frameworks and/or one or more VL frameworks. The antibody generated by introducing substitutions in the VH domain, VH CDRs, VL domain, VL CDRs and/or frameworks of 4D4, 4D4H2-1 D11, 4D4com-XF-9, 4D4com-2F9, 7F3, 71A10, 7F3 22D3, 7F3com-2H2, 7F3com-3H5, or 7F3com-3D4 can be tested in vitro and/or in vivo, for example, for its ability to bind to an IL-9 polypeptide, or for its ability to inhibit or reduce IL-9 mediated cell proliferation, or for its ability to prevent, treat and/or ameliorate a fibrotic or inflammatory disease or disorder or a symptom thereof.

The antibody that immunospecifically binds IL-9 polypeptide may comprise an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of 4D4, 4D4H2-1 D11, 4D4com-XF-9, 4D4com-2F9, 7F3, 71A10, 7F3 22D3, 7F3com-2H2, 7F3com-3H5 or 7F3com-3D4, or an antigen-binding fragment thereof. In another embodiment, an antibody that immunospecifically binds to an IL-9 polypeptide comprises an amino acid sequence of a VH domain that is at least 35%, preferably at least 40%, at least 45%, at least 50%, at least 55%, at least 60%, at least 65%, at least 70%, at least 75%, at least 80%, at least 85%, at least 90%, at least 95%, or at least 99% identical to the VH domain of 4D4, 4D4H2-1 D11, 4D4com-XF-9, 4D4com-2F9, 7F3, 71A10, 7F3 22D3, 7F3com-2H2, 7F3com-3H5 or 7F3com-3D4. In another embodiment, an antibody that immunospecifically binds to an IL-9 polypeptide comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of 4D4, 4D4H2-1 D11, 4D4com-XF-9, 4D4com-2F9, 7F3, 71A10, 7F3 22D3, 7F3com-2H2, 7F3com-3H5 or 7F3com-3D4.

The antibody that immunospecifically binds IL-9 may comprise an amino acid sequence of one or more VL CDRs (VL CDR1, VL CDR2, VL CDR3, VL CDR1 and CDR2, VL CDR1 and CDR3, VL CDR2 and CDR3, or VL CDR1, CDR2, and CDR3) that are at least 35%, at least 40%, at least 45%, at least 50%, at least 55%, at least 60%, at least 65%, at least 70%, at least 75%, at least 80%, at least 85%, at least 90%, at least 95%, or at least 99% identical to VL CDRs of any one of antibodies 4D4, 4D4H2-1 D11, 4D4com-XF-9, 4D4com-2F9, 7F3, 71A10, 7F3 22D3, 7F3com-2H2, 7F3com-3H5 or 7F3com-3D4. The antibody that immunospecifically binds IL-9 may comprise an amino acid sequence of one or more VH CDRs (VH CDR1, VH CDR2, VH CDR3, VH CDR1 and CDR2, VH CDR1 and CDR3, VH CDR2 and CDR3, or VH CDR1, CDR2, and CDR3) that are at least 35%, preferably at least 40%, at least 45%, at least 50%, at least 55%, at least 60%, at least 65%, at least 70%, at least 75%, at least 80%, at least 85%, at least 90%, at least 95%, or at least 99% identical to any of one of the VH CDRs of antibodies 4D4, 4D4H2-1 D11, 4D4com-XF-9, 4D4com-2F9, 7F3, 71A10, 7F3 22D3, 7F3com-2H2, 7F3com-3H5 or 7F3com-3D4. The VL CDR(s) may be VL CDR1, or VL CDR2, or VL CDR3, or VL CDR 1 and 2, or VL CDR 2 and 3, or VL CDR 1 and 3, or VL CDR 1, 2, and 3 of any one of antibodies 4D4, 4D4H2-1 D11, 4D4com-XF-9, 4D4com-2F9, 7F3, 71A10, 7F3 22D3, 7F3com-2H2, 7F3com-3H5 or 7F3com-3D4. The VH CDR(s) may be VH CDR1, or VH CDR2, or VH CDR3, or VH CDR 1 and 2, or VH CDR 2 and 3, or VH CDR 1 and 3, or VH CDR 1, 2, and 3 of any one of antibodies 4D4, 4D4H2-1 D11, 4D4com-XF-9, 4D4com-2F9, 7F3, 71A10, 7F3 22D3, 7F3com-2H2, 7F3com-3H5 or 7F3com-3D4. The determination of percent identity between two sequences can also be accomplished using a mathematical algorithm. A non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. The percent identity between two sequences can be determined using techniques similar to those described above, with or without allowing gaps. In calculating percent identity, only exact matches may be counted.

The antibodies that specifically bind IL-9 may compete with any one of 4D4, 4D4H2-1 D11, 4D4com-XF-9, 4D4com-2F9, 7F3, 71A10, 7F3 22D3, 7F3com-2H2, 7F3com-3H5 or 7F3com-3D4 for binding to IL-9. The antibody that specifically binds IL-9 may reduce binding of 4D4, 4D4H2-1 D11, 4D4com-XF-9, 4D4com-2F9, 7F3, 71A10, 7F3 22D3, 7F3com-2H2, 7F3com-3H5 or 7F3com-3D4 to IL-9 by at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more, 25% to 50%, 45 to 75%, or 75 to 99%. Competitve binding may be determined by an ELISA competition assay performed in the following manner: recombinant IL-9 is prepared in PBS at a concentration of 10 μg/ml. 100 μl of this solution is added to each well of an ELISA 98-well microtiter plate and incubated overnight at 4-8° C. The ELISA plate is washed with PBS supplemented with 0.1% Tween to remove excess recombinant IL-9. Non-specific protein-protein interactions are blocked by adding 100 μl of bovine serum albumin (BSA) prepared in PBS to a final concentration of 1%. After one hour at room temperature, the ELISA plate is washed. Unlabeled competing antibodies are prepared in blocking solution at concentrations ranging from 1 μg/ml to 0.01 μg/ml. Control wells contain either blocking solution only or control antibodies at concentrations ranging from 1 μg/ml to 0.01 μg/ml. Test antibody (e.g., 7F3com-2H2) labeled with horseradish peroxidase is added to competing antibody dilutions at a fixed final concentration of 1 μg/ml. 100 μl of test and competing antibody mixtures are added to the ELISA wells in triplicate and the plate is incubated for 1 hour at room temperature. Residual unbound antibody is washed away. Bound test antibody is detected by adding 100 μl of horseradish peroxidase substrate to each well. The plate is incubated for 30 min. at room temperature, and absorbance is read using an automated plate reader. The average of triplicate wells is calculated. Antibodies which compete well with the test antibody reduce the measured absorbance compared with control wells.

The antibodies that immunospecifically bind IL-9 may be modified, i.e., by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the modified antibodies may contain one or more non-classical amino acids.

The antibodies that immunospecifically bind IL-9 may comprise any framework region known to those of skill in the art (e.g., a human or non-human framework). The framework regions may be naturally occurring or consensus framework regions. Preferably, the fragment region of an antibody of the invention is human (see, e.g., Chothia et al., 1998, J. Mol. Biol. 278:457-479 for a listing of human framework regions, which is incorporated herein by reference in its entirety).

The antibodies that immunospecifically bind IL-9 may comprise the amino acid sequence of 4D4, 4D4H2-1 D11, 4D4com-XF-9, 4D4com-2F9, 7F3, 71A10, 7F3 22D3, 7F3com-2H2, 7F3com-3H5 or 7F3com-3D4 with mutations (e.g., one or more amino acid substitutions) in the framework regions. The antibodies may comprise the amino acid sequence of 4D4, 4D4H2-1 D11, 4D4com-XF-9, 4D4com-2F9, 7F3, 71A10, 7F3 22D3, 7F3com-2H2, 7F3com-3H5 or 7F3com-3D4 with one or more amino acid residue substitutions in the framework regions of the VH and/or VL domains. The amino acid substitutions in the framework region may be made to improve binding of the antibody to IL-9.

The antibodies that immunospecifically bind IL-9 may comprise the amino acid sequence of 4D4, 4D4H2-1 D11, 4D4com-XF-9, 4D4com-2F9, 7F3, 71A10, 7F3 22D3, 7F3com-2H2, 7F3com-3H5 or 7F3com-3D4 with mutations (e.g., one or more amino acid residue substitutions) in the variable and framework regions. The amino acid substitutions in the variable and framework regions may be made to improve binding of the antibody to IL-9. The antibodies that specifically bind IL-9 may comprise any constant regions known to those of skill in the art. The constant regions of the antibody may be human.

Further antibodies that immunospecifically bind IL-9 may be produced by any method known in the art for the synthesis of antibodies, in particular, by chemical synthesis or by recombinant expression techniques.

Polyclonal antibodies that immunospecifically bind to IL-9 can be produced by various procedures well-known in the art. For example, IL-9 polypeptide can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with an IL-9 polypeptide and once an immune response is detected, e.g., antibodies specific for IL-9 are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. Additionally, a RIMMS (repetitive immunization multiple sites) technique can be used to immunize an animal (Kilptrack et al., 1997 Hybridoma 16:381-9, incorporated by reference in its entirety). The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Antibody fragments which recognize specific IL-9 epitopes may be generated by any technique known to those of skill in the art. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make IL-9 antibodies include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/O1 134; International Publication Nos. WO 90/02809, WO 91/10737, WO 92/01047, WO 92/18619, WO 93/1 1236, WO 95/15982, WO 95/20401, and W097/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)₂ fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. The vectors for expressing the VH or VL domains may comprise an EF-1α promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors can then be co-transfected into cell lines to generate stable or transient cell lines that express full-length antibodies, e.g., IgG, using techniques known to those of skill in the art.

The antibodies that specifically bind IL-9 may be human or chimeric antibodies. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO98/16654, WO 96/34096, WO 96/33735, and WO 91/10741.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of technology for producing human antibodies and human monoclonal antibodies and protocols for producing such antibodies, see, e.g., PCT publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and two, variable domains (Fab, Fab′, F(ab′)₂, Fabc, Fv) in which all or substantially all of the CDR regions correspond to those of a non-human immunoglobulin (i.e., donor antibody) and all or substantially all of the framework regions are those of a human immunoglobulin consensus sequence. A humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG₁. Where such cytotoxic activity is not desirable, the constant domain may be of the IgG₂ class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental FR and CDR sequences, or at least 90%, or at least 95%. Humanized antibody can be produced using variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119-25 (2002), Caldas et al., Protein Eng. 13(5):353-60 (2000), Morea et al., Methods 20(3):267-79 (2000), Baca et al., J. Biol. Chem. 272(16):10678-84 (1997), Roguska et al., Protein Eng. 9(10):895-904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). Framework residues in the framework regions may be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323.)

Single domain antibodies, for example, antibodies lacking the light chains, can be produced by methods well-known in the art. See Riechmann et al., 1999, J. Immuno. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and International Publication Nos. WO 94/04678, WO 94/25591, and WO 01/44301.

Antibodies immunospecific for IL-9 that are produced by any of the methods discussed above or otherwise known to those of skill in the art may be characterized in a variety of ways. For instance, antibodies immunospecific for IL-9 may be assayed for the ability to immunospecifically bind to an IL-9 polypeptide. Such an assay may be performed in solution (e.g., Houghten, 1992, Bio/Techniques 13:412-421), on beads (Lam, 1991, Nature 354:82-84), on chips (Fodor, 1993, Nature 364:555-556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865-1869) or on phage (Scott and Smith, 1990, Science 249:386-390; Cwirla et al., 1990, Proc. Natl. Acad. Sci. USA 87:6378-6382; and Felici, 1991, J. Mol. Biol. 222:301-310). Antibodies that have been identified to immunospecifically bind IL-9 can then be assayed for their specificity and affinity for an IL-9 polypeptide.

IL-9 antibodies may be assayed for immunospecific binding to an IL-9 polypeptide and cross-reactivity with other antigens by any method known in the art. Immunoassays which can be used to analyze immunospecific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1 to 4 hours) at 40° C., adding protein A and/or protein G sepharose beads to the cell lysate, incubating for about an hour or more at 40° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, incubating the membrane in blocking solution (e.g., PBS with 3% BSA or non-fat milk), washing the membrane in washing buffer (e.g., PBS-Tween 20), incubating the membrane with primary antibody (the antibody of interest) diluted in blocking buffer, washing the membrane in washing buffer, incubating the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. In a preferred embodiment, an ELISA may be performed by coating a high binding 96-well microtiter plate (Costar) with 2 .mu.g/ml of rhu-IL-9 in PBS overnight. Following three washes with PBS, the plate is incubated with three-fold serial dilutions of Fab at 25° C. for 1 hour. Following another three washes of PBS, 1 μg/ml anti-human kappa-alkaline phosphatase-conjugate is added and the plate is incubated for 1 hour at 25° C. Following three washes with PBS, the alkaline phosphatase activity is determined in 50 μl/AMP/PPMP substrate. The reactions are stopped and the absorbance at 560 nm is determined with a VMAX microplate reader. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of the present invention or a fragment thereof for an IL-9 polypeptide and the binding off-rates can be determined from the data by scatchard plot analysis. Competition with a second antibody can also be determined using radioimmunoassays. In this case, an IL-9 polypeptide is incubated with an antibody of the present invention conjugated to a labeled compound (e.g., ³H or ¹²⁵I in the presence of increasing amounts of an unlabeled second antibody.

BIAcore kinetic analysis may be used to determine the binding on and off rates of antibodies immunospecific for IL-9. BIAcore kinetic analysis comprises analyzing the binding and dissociation of an IL-9 polypeptide from chips with immobilized antibodies of the invention on their surface. A typical BIAcore kinetic study involves the injection of 250 μL of an antibody reagent (mAb, Fab) at varying concentration in HBS buffer containing 0.005% Tween-20 over a sensor chip surface, onto which has been immobilized the antigen. The flow rate is maintained constant at 75 μL/min. Dissociation data is collected for 15 min. or longer as necessary. Following each injection/dissociation cycle, the bound mAb is removed from the antigen surface using brief, 1 min. pulses of dilute acid, typically 10-100 mM HCl, though other regenerants are employed as the circumstances warrant. More specifically, for measurement of the rates of association, kₒₙ, and dissociation, k_{off}, the antigen is directly immobilized onto the sensor chip surface through the use of standard amine coupling chemistries, namely the EDC/NHS method (EDC=N-diethylaminopropyl)-carbodiimide). Briefly, a 5-100 r1M solution of the antigen in 10 mM NaOAc, pH4 or pH5 is prepared and passed over the EDC/NHS-activated surface until approximately 30-50 RU's worth of antigen are immobilized. Following this, the unreacted active esters are “capped” off with an injection of 1M Et-NH2. A blank surface, containing no antigen, is prepared under identical immobilization conditions for reference purposes. Once an appropriate surface has been prepared, a suitable dilution series of each one of the antibody reagents is prepared in HBS/Tween-20, and passed over both the antigen and reference cell surfaces, which are connected in series. The range of antibody concentrations that are prepared varies, depending on what the equilibrium binding constant, KD, is estimated to be. As described above, the bound antibody is removed after each injection/dissociation cycle using an appropriate regenerant.

Antibodies specific for IL-9 can also be assayed for their ability to inhibit the binding of IL-9 to its host cell receptor using further techniques known to those of skill in the art. For example, cells expressing IL-9 receptor can be contacted with IL-9 in the presence or absence of an antibody or fragment thereof and the ability of the antibody or fragment thereof to inhibit IL-9's binding can measured by, for example, flow cytometry or a scintillation assay. IL-9 or the antibody or antibody fragment can be labeled with a detectable compound such as a radioactive label (e.g., ³²P, ³⁵S, and ¹²⁵I) or a fluorescent label (e.g., fluorescein isothiocyanate, rhodamine, phycoerythrin, phycocyanin, allophycocyanin, o-phthaldehyde and fluorescamine) to enable detection of an interaction between IL-9 and its host cell receptor. Alternatively, the ability of antibodies or fragments thereof to inhibit IL-9 from binding to its receptor can be determined in cell-free assays. For example, an IL-9 polypeptide can be contacted with an antibody or fragment thereof and the ability of the antibody or antibody fragment to inhibit the IL-9 polypeptide from binding to its host cell receptor can be determined. The antibody or the antibody fragment is immobilized on a solid support and an IL-9 polypeptide is labeled with a detectable compound. Alternatively, an IL-9 polypeptide is immobilized on a solid support and the antibody or fragment thereof is labeled with a detectable compound. An IL-9 may be partially or completely purified (e.g., partially or completely free of other polypeptides) or part of a cell lysate. Further, an IL-9 polypeptide may be a fusion protein comprising IL-9, a derivative, analog or fragment thereof and a domain such as glutathionine-S-transferase. Alternatively, an IL-9 polypeptide can be biotinylated using techniques well known to those of skill in the art (e.g., biotinylation kit, Pierce Chemicals; Rockford, Ill.).

The ability of antibodies or fragments to inhibit IL-9 from binding to a host cell receptor may be measured by cell proliferation assays. For example, the murine TS1-RA3 T cell line expressing both human and murine IL-9Ra may be grown continuously in growth medium (DMEM) containing rhuIL-9 (25 ng/ml, R & D Systems). Upon withdrawal of rhuIL-9, TS 1-RA3 undergoes cell death in 18-24 hours. TS 1-RA3 cells grown in RPMI 1640 (ATCC) medium supplemented with 10% FBS and 25 ng/ml rHu-IL9. Prior to the assay, the cells are washed with media containing no IL-9 and resuspended at 5×10⁵ cells/ml in media containing 2 ng/ml rhuIL-9. The cells are distributed into a black clear bottom non-binding 96-well microtiter plate (100 μl cells/well) and 100 ml of serially diluted variant Fabs is then added to the plate. The plate is incubated at 72 hours at 37° C., 5% CO₂. 20 μl/well of Alamar blue® is added, and the cells are incubated for an additional 4-5 hours. Cell metabolism is quantitated using a fluorimeter with excitation at 555 nm and emission at 590 nm. The ability of antibodies or fragments of the invention to inhibit IL-9 from binding to its host cell receptor may be measured may also be measured by a cell binding assay, such as an IL-9 binding ELISA assay. For example, each well of a 96-well ELISA plate is coated with 100 μL of IL-9 antibodies or antibody fragments of the invention overnight at 2 to 8° C. The plate is washed three times with PBS/0.5% Tween-20 buffer, and is blocked for 1 hour at ambient temperature with PBS/0.1% Tween-20 buffer, 1% (w/v) BSA. After washing the plate, 100 μL of a Reference Standard, samples and controls are loaded onto the assay plate and incubated at ambient temperature for 1 hour. After washing, 100 μL of horseradish peroxidase-labeled (HRP) goat anti-human IgG at a 1:15,000 dilution is added to the assay plate. Following the one-hour incubation, the plate is washed and 100 .mu.L/well of 3,3′,5,5′-tetramethylbenzidine (TMB) substrate is added to the plate and incubated at ambient temperature in the dark for 10 minutes. The enzymatic reaction is stopped by the addition of 50 μL/well of 2N sulfuric acid. The absorbance at 450 nm is measured using a microplate reader. Samples are dispositioned as pass/fail based on the parallelism of the sample curve to the Reference Standard curve, and the ED₅₀ value of the sample falling in the range of 3.91-31.91 ng/mL.

Small Molecule Inhibitors of IL-9

The inhibitors of IL-9 employed by the methods encompassed by invention may be small molecule inhibitors of IL-9. The small molecule inhibitors of IL-9 include proteins, naturally occurring proteins or fragments of naturally occurring proteins, or artificially derived proteins Inhibitors of IL-9 may be peptides of from about 5 to about 30 amino acids, or about 5 to about 20 amino acids, or about 7 to about 15 amino acids. The peptides may be digests of naturally occurring proteins, or random peptides, or “biased” random peptides. Randomized peptides are essentially random sequences of amino acids which are often chemically synthesized. These peptides can be produced by a synthetic process designed to generate the formation of all or most of the possible combinations of amino acid residues over the length of a sequence. Such a library has no sequence preferences or constants over any amino acid residue at any position. Alternatively, the library may comprise biased randomized peptides, i.e., some residue positions within the amino acid sequence are either held constant, or are selected from a limited number of possibilities.

The small molecule may be a chemical compound. The small molecule chemical compound inhibitor of IL-9 may be identified from an existing or newly synthesized combinatorial chemical library. A combinatorial chemical library is a collection of diverse chemical compounds generated by chemical synthesis by combining a number of chemical “building blocks” such as reagents. For instance, millions of chemical compounds can be synthesized through such combinatorial mixing of chemical building blocks. Gallop et al., J. Med. Chem. 37(9): 1233-1251 (1994).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. The above devices, with appropriate modification, are suitable for preparing combinatorial libraries. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

In general, combinatorial chemical libraries are well known to those of skill in the art. Such combinatorial chemical libraries include, but are not limited to, peptide libraries (see, e.g., U.S. Pat. No. 5,010,175, Furka, Pept. Prot. Res. 37: 487-493 (1991), Houghton et al., Nature, 354: 84-88 (1991)), peptoids (PCT Publication No. WO 91/19735), encoded peptides (PCT Publication No. WO 93/20242), random bio-oligomers (PCT Publication WO 92/00091), benzodiazepines (U.S. Pat. No. 5,288,514), diversomers such as hydantoins, benzodiazepines and dipeptides (Hobbs et al., Proc. Nat. Acad. Sci. USA 90: 6909-6913 (1993)), vinylogous polypeptides (Hagihara et al., J. Amer. Chem. Soc. 114: 6568 (1992)), nonpeptidal peptidomimetics with a Beta-D-Glucose scaffolding (Hirschmann et al., J. Amer. Chem. Soc. 114: 9217-9218 (1992)), analogous organic syntheses of small compound libraries (Chen et al., J. Amer. Chem. Soc. 116: 2661 (1994)), oligocarbamates (Cho, et al., Science 261: 1303 (1993)), and/or peptidyl phosphonates (Campbell et al., J. Org. Chem. 59: 658 (1994)). See also, generally, Gordon et al., J. Med. Chem. 37: 1385 (1994), nucleic acid libraries (see, e.g., Stratagene, Corp.), peptide nucleic acid libraries (see, e.g., U.S. Pat. No. 5,539,083), antibody libraries (see, e.g., Vaughn et al., Nature Biotechnology 14(3: 309-314 (1996), and PCT/US96/10287), carbohydrate libraries (see, e.g., Liang et al., Science 274: 1520-1522 (1996), and U.S. Pat. No. 5,593,853), and small organic molecule libraries (see, e.g., benzodiazepines, Baum, C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514; and the like). Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

Small molecule inhibitors of IL-9 may be identified from libraries by screening. High throughput screening (HTS) methods may be employed for a screening analysis. High throughput screening methods may involve providing a library containing a large number of small molecules and then screening in one or more assays to identify those library members (particular chemical species or subclasses) that display a desired characteristic activity, e.g., inhibiting interaction of IL-9 with IL-9 receptor. The compounds identified can serve as conventional lead compounds or can themselves be used as potential or actual therapeutics. For detection of IL-9 interactions, assays that detect IL-9-mediated signal transduction may be used such as IL-9-mediated phosphorylation of JAK1 and JAK3 tyrosine kinases. Other high throughput assays for evaluating the presence, absence, quantification, or other properties of particular polypeptides are well known to those of skill in the art.

Furthermore, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate procedures, including sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, e.g., Zymark Corp. provides technical bulletins describing screening systems for detecting the modulation of gene transcription, ligand binding, and the like.

Small molecules that may be employed in the methods of the invention may be monobodies, or antibody mimics. These molecules comprise peptide domains, e.g., fibronectin domains, which may be designed to bind a molecule of interest, e.g., IL-9 or IL-9 receptor. Description of such domains, production of such domains, and screening of such domains can be found in, for example, U.S. Pat. Nos. 6,673,901, 7,153,661, and U.S. patent application publications 2005-0048512 and 2006-0223114.

Other small molecules that may be employed in the methods of the invention may be soluble IL-9 receptor polypeptides. Soluble IL-9 receptor polypeptides have been described in U.S. Pat. No. 6,602,850.

Yet other small molecules that may be employed in the method of the invention may be chemical compounds. Such chemical compounds include, e.g., 3-aminosteriod compounds. See, e.g., U.S. Pat. No. 7,074,778.

### A Therapeutic Agent Which is Not an Inhibitor of IL-9

The methods encompassed by invention may employ, in addition to antibodies specific for IL-9, a therapeutic agent which is not an antibody specific for IL-9. Such therapeutic agents may include, but are not limited to, small molecules, synthetic drugs, peptides, polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides) antibodies, synthetic or natural inorganic molecules, mimetic agents, and synthetic or natural organic molecules.

Any therapeutic agent which is known to be useful, or which has been used or is currently being used for the prevention, management, treatment, or amelioration of one or more symptoms associated with a disorder can be used in combination with an antibody that immunospecifically binds IL-9. See, e.g., Gilman et al., Goodman and Gilman's: The Pharmacological Basis of Therapeutics, Tenth Ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; and Cecil Textbook of Medicine, 20th Ed., Bennett and Plum (eds.), W. B. Saunders, Philadelphia, 1996 for information regarding therapies, in particular prophylactic or therapeutic agents, which have been or are currently being used for preventing, treating, managing, and/or ameliorating disorders. Examples of therapeutic agents include, but are not limited to, immunomodulatory agents, anti-inflammatory agents (e.g., adrenocorticoids, corticosteroids (e.g., beclomethasone, budesonide, flunisolide, fluticasone, triamcinolone, methlyprednisolone, prednisolone, prednisone, hydrocortisone), glucocorticoids, steroids, non-steriodal anti-inflammatory drugs (e.g., aspirin, ibuprofen, diclofenac, and COX-2 inhibitors), and leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents, and antibiotics (e.g., dactinomycin (formerly actinomycin), bleomycin, erythomycin, penicillin, mithramycin, and anthramycin (AMC)).

Immunomodulatory Therapeutic Agents

Any immunomodulatory agent well-known to one of skill in the art may be used in the methods encompassed by the invention. Immunomodulatory agents can affect one or more or all aspects of the immune response in a subject. Aspects of the immune response include, but are not limited to, the inflammatory response, the complement cascade, leukocyte and lymphocyte differentiation, proliferation, and/or effector function, monocyte and/or basophil counts, and the cellular communication among cells of the immune system. In certain embodiments of the invention, an immunomodulatory agent modulates one aspect of the immune response. An immunomodulatory agent may modulate more than one aspect of the immune response. The immunomodulatory agent may inhibit or reduce one or more aspects of a subject's immune response capabilities. The immunomodulatory agent may inhibit or suppress the immune response of a subject.

Examples of immunomodulatory agents include, but are not limited to, proteinaceous agents such as cytokines, peptide mimetics, and antibodies (e.g., human, humanized, chimeric, monoclonal, polyclonal, Fvs, ScFvs, Fab or F(ab)₂ fragments or epitope binding fragments), nucleic acid molecules (e.g., antisense nucleic acid molecules and triple helices), small molecules, organic compounds, and inorganic compounds. Immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), T cell receptor modulators, cytokine receptor modulators, and modulators mast cell modulators.

Examples of T cell receptor modulators include, but are not limited to, anti-T cell receptor antibodies (e.g., anti-CD4 antibodies (e.g., cM-T412 (Boeringer), IDEC-CE9.1®. (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies (e.g., siplizumab (MedImmune, Inc., International Publication Nos. WO 02/098370 and WO 02/069904)), anti-CD11a antibodies (e.g., Xanelim (Genentech)), and anti-B7 antibodies (e.g., IDEC-114) (IDEC))), CTLA4-immunoglobulin, and LFA-3TIP (Biogen, International Publication No. WO 93/08656 and U.S. Pat. No. 6,162,432).

Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g., interleukin IL-2, IL-3, IL-4, IL-5, IL-6, IL-7, IL-8, IL-9, IL-10, IL-11, IL-12, IL-13, IL-15, IL-23, TNF-α, TNF-β, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-3 receptor antibodies, anti-IL-4 receptor antibodies, anti-IL-6 receptor antibodies, anti-IL-10 receptor antibodies, anti-IL-12 receptor antibodies, anti-IL-13 receptor antibodies, anti-IL-15 receptor antibodies, and anti-IL-23 receptor antibodies), anti-cytokine antibodies (e.g., anti-IFN antibodies, anti-TNF-α antibodies, anti-IL-10 antibodies, anti-IL-3 antibodies, anti-IL-6 antibodies, anti-IL-8 antibodies (e.g., ABX-IL-8 (Abgenix)), anti-IL-12 antibodies, anti-IL-13 antibodies, anti-IL-15 antibodies, and anti-IL-23 antibodies).

A cytokine receptor modulator may be a mast cell modulator. Alternatively, a cytokine receptor modulator may not be a mast cell modulator. Examples of mast cell modulators include, but are not limited to stem cell factor (c-kit receptor ligand) inhibitors (e.g., mAb 7H6, mAb 8H7a, pAb 1337, FK506, CsA, dexamthasone, and fluconcinonide), c-kit receptor inhibitors (e.g., STI 571 (formerly known as CGP 57148B)), mast cell protease inhibitors (e.g., GW-45, GW-58, wortmannin, LY 294002, calphostin C, cytochalasin D, genistein, KT5926, staurosproine, and lactoferrin), relaxin (“RLX”), IgE antagonists (e.g., antibodies rhuMAb-E25 omalizumab, HMK-12 and 6HD5, and mAB Hu-901), IL-3 antagonists, IL-4 antagonists, IL-10 antagonists, and TGF-beta.

An immunomodulatory agent may be selected to interfere with the interactions between the T helper subsets (TH1 or TH2) and B cells to inhibit neutralizing antibody formation. Antibodies that interfere with or block the interactions necessary for the activation of B cells by TH (T helper) cells, and thus block the production of neutralizing antibodies, may be used as immunomodulatory agents in the methods of the invention. For example, B cell activation by T cells requires certain interactions to occur (Durie et al., Immunol. Today, 15(9):406-410 (1994)), such as the binding of CD40 ligand on the T helper cell to the CD40 antigen on the B cell, and the binding of the CD28 and/or CTLA4 ligands on the T cell to the B7 antigen on the B cell. Without both interactions, the B cell cannot be activated to induce production of the neutralizing antibody.

The CD40 ligand (CD4OL)-CD40 interaction may be employed in the methods encompassed by the invention to block the immune response. This can be accomplished by treating with an agent which blocks the CD40 ligand on the TH cell and interferes with the normal binding of CD40 ligand on the T helper cell with the CD40 antigen on the B cell. An antibody to CD40 ligand (anti-CD4OL) (available from Bristol-Myers Squibb Co; see, e.g., European patent application 555,880, published Aug. 18, 1993) or a soluble CD40 molecule can be selected and used as an immunomodulatory agent.

An immunomodulatory agent may be selected to inhibit the interaction between TH1 cells and cytotoxic T lymphocytes (“CTLs”) to reduce the occurrence of CTL-mediated killing. An immunomodulatory agent may be selected to alter (e g , inhibit or suppress) the proliferation, differentiation, activity and/or function of the CD4⁺ and/or CD8⁻ T cells. For example, antibodies specific for T cells can be used as immunomodulatory agents to deplete, or alter the proliferation, differentiation, activity and/or function of CD4⁺ and/or CD8⁻ T cells.

An immunomodulatory agent may reduce or deplete T cells. An immunomodulatory agent may inactivate CD8⁺ T cells, e.g., anti-CD8 antibodies may be used to reduce or deplete CD8⁺ T cells.

In another embodiment, an immunomodulatory agent may reduce or inhibit one or more biological activities (e.g., the differentiation, proliferation, and/or effector functions) of TH0, TH1, and/or TH2 subsets of CD4⁺ T helper cells. One example of such an immunomodulatory agent is IL-4. IL-4 enhances antigen-specific activity of TH2 cells at the expense of the TH1 cell function (see, e.g., Yokota et al, 1986 Proc. Natl. Acad. Sci., USA, 83:5894-5898; and U.S. Pat. No. 5,017,691). Other examples of immunomodulatory agents that affect the biological activity (e.g., proliferation, differentiation, and/or effector functions) of T-helper cells (in particular, TH1 and/or TH2 cells) include, but are not limited to, IL-2, IL-4, IL-5, IL-6, IL-10, IL-12, IL-13, IL-15, IL-23, and interferon (IFN)-γ.

An immunomodulatory agent may be a cytokine that prevents antigen presentation. Such a cytokine may be IL-10. IL-10 also reduces or inhibits macrophage action which involves bacterial elimination.

An immunomodulatory agent may be an agent which reduces or inhibits the activation, degranulation, proliferation, and/or infiltration of mast cells. These agents include, but are not limited to stem cell factors (c-kit ligands), IgE, IL-4, environmental irritants, and infectious agents. The immunomodulatory agent may reduce or inhibit the response of mast cells to environmental irritants such as, but not limited to pollen, dust mites, tobacco smoke, and/or pet dander. The immunomodulatory agent may reduce or inhibit the response of mast cells to infectious agents, such as viruses, bacteria, and fungi. Examples of mast cell modulators that reduce or inhibit the activation, degranulation, proliferation, and/or infiltration of mast cells include, but are not limited to, stem cell factor (c-kit receptor ligand) inhibitors (e.g., mAb 7H6, mAb 8H7a, and pAb 1337 (see Mendiaz et al., 1996, Eur J Biochem 293(3):842-849), FK506 and CsA (Ito et al., 1999 Arch Dermatol Res 291(5):275-283), dexamthasone and fluconcinonide (see Finooto et al. J Clin Invest 1997 99(7): 1721-1728)), c-kit receptor inhibitors (e.g., STI 571 (formerly known as CGP 57148B) (see Heinrich et al., 2000 Blood 96(3):925-932)), mast cell protease inhibitors (e.g., GW-45 and GW-58 (see Temkin et al., 2002 J Immunol 169(5):2662-2669), wortmannin, LY 294002, calphostin C, and cytochalasin D (see Vosseller et al., 1997, Mol Biol Cell 1997: 909-922), genistein, KT5926, and staurosproine (see Nagai et al. 1995, Biochem Biophys Res Commun 208(2):576-581), and lactoferrin (see He et al., 2003 Biochem Pharmacol 65(6):1007-1015)), relaxin (“RLX”) (see Bani et al., 2002 Int Immunopharmacol 2(8):1195-1294),), IgE antagonists (e.g., antibodies rhuMAb-E25 omalizumab (see Finn et al., 2003 J Allergy Clin Immuno 111(2):278-284; Corren et al., 2003 J Allergy Clin Immuno 111(1):87-90; Busse and Neaville, 2001 Curr Opin Allergy Clin Immuno 1(1):105-108; and Tang and Powell, 2001, Eur J Pediatr 160(12): 696-704), HMK-12 and 6HD5 (see Miyajima et al., 2202 Int Arch Allergy Immuno 128(1):24-32), and mAB Hu-901 (see van Neerven et al., 2001 Int Arch Allergy Immuno 124(1-3):400), IL-3 antagonist, IL-4 antagonists, IL-10 antagonists, and TGF-beta (see Metcalfe et al., 1995, Exp Dermatol 4(4 Pt 2):227-230).

Anti-Angiogenic Therapies

Any anti-angiogenic agent well-known to one of skill in the art may be used in the methods encompassed by the invention. Non-limiting examples of anti-angiogenic agents include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that immunospecifically bind to TNF-α, nucleic acid molecules (e.g., antisense molecules or triple helices), organic molecules, inorganic molecules, and small molecules that reduce or inhibit angiogenesis. In particular, examples of anti-angiogenic agents, include, but are not limited to, endostatin, angiostatin, apomigren, anti-angiogenic antithrombin III, the 29 kDa N-terminal and a 40 kDa C-terminal proteolytic fragments of fibronectin, a uPA receptor antagonist, the 16 kDa proteolytic fragment of prolactin, the 7.8 kDa proteolytic fragment of platelet factor-4, the anti-angiogenic 24 amino acid fragment of platelet factor-4, the anti-angiogenic factor designated 13.40, the anti-angiogenic 22 amino acid peptide fragment of thrombospondin I, the anti-angiogenic 20 amino acid peptide fragment of SPARC, RGD and NGR containing peptides, the small anti-angiogenic peptides of laminin, fibronectin, procollagen and EGF, integrin αᵥβ₃ antagonists, acid fibroblast growth factor (aFGF) antagonists, basic fibroblast growth factor (bFGF) antagonists, vascular endothelial growth factor (VEGF) antagonists (e.g., anti-VEGF antibodies), and VEGF receptor (VEGFR) antagonists (e.g., anti-VEGFR antibodies).

Examples of integrin αᵥβ₃ antagonists include, but are not limited to, proteinaceous agents such as non-catalytic metalloproteinase fragments, RGD peptides, peptide mimetics, fusion proteins, disintegrins or derivatives or analogs thereof, and antibodies that immunospecifically bind to integrin αᵥβ₃, nucleic acid molecules, organic molecules, and inorganic molecules. Non-limiting examples of antibodies that immunospecifically bind to integrin αᵥβ₃ include 11D2 (Searle), LM609 (Scripps), and VITAXIN™ (MedImmune, Inc.). Non-limiting examples of small molecule peptidometric integrin αᵥβ₃ antagonists include S836 (Searle) and S448 (Searle). Examples of disintegrins include, but are not limited to, Accutin. The invention also encompasses the use of any of the integrin αᵥβ₃ antagonists disclosed in the following U.S. patents and International publications in the compositions and methods of the invention: U.S. Pat. Nos. 5,149,780; 5,196,511; 5,204,445; 5,262,520; 5,306,620; 5,478,725; 5,498,694; 5,523,209; 5,578,704; 5,589,570; 5,652,109; 5,652,110; 5,693,612; 5,705,481; 5,753,230; 5,767,071; 5,770,565; 5,780,426; 5,817,457; 5,830,678; 5,849,692; 5,955,572; 5,985,278; 6,048,861; 6,090,944; 6,096,707; 6,130,231; 6,153,628; 6,160,099; and 6,171,588; and International Publication Nos. WO 95/22543; WO 98/33919; WO 00/78815; and WO 02/070007.

An anti-angiogenic agent may endostatin. Naturally occurring endostatin consists of the C-terminal about 180 amino acids of collagen XVIII (cDNAs encoding two splice forms of collagen XVIII have GenBank Accession Nos. AF18081 and AF18082). An anti-angiogenic agent may be a plasminogen fragment (the coding sequence for plasminogen can be found in GenBank Accession Nos. NM_{—}000301 and A33096). Angiostatin peptides naturally include the four kringle domains of plasminogen, kringle 1 through kringle 4. It has been demonstrated that recombinant kringle 1, 2 and 3 possess the anti-angiogenic properties of the native peptide, whereas kringle 4 has no such activity (Cao et al., 1996, J. Biol. Chem. 271:29461-29467). An angiostatin peptide may comprise at least one or more than one kringle domain selected from the group consisting of kringle 1, kringle 2 and kringle 3. An anti-angiogenic peptide may be the 40 kDa isoform of the human angiostatin molecule, the 42 kDa isoform of the human angiostatin molecule, the 45 kDa isoform of the human angiostatin molecule, or a combination thereof. An anti-angiogenic agent may be the kringle 5 domain of plasminogen. An anti-angiogenic agent may be antithrombin III. Antithrombin III, which is referred to hereinafter as antithrombin, comprises a heparin binding domain that tethers the protein to the vasculature walls, and an active site loop which interacts with thrombin. When antithrombin is tethered to heparin, the protein elicits a conformational change that allows the active loop to interact with thrombin, resulting in the proteolytic cleavage of said loop by thrombin. The proteolytic cleavage event results in another change of conformation of antithrombin, which (i) alters the interaction interface between thrombin and antithrombin and (ii) releases the complex from heparin (Carrell, 1999, Science 285:1861-1862, and references therein). O'Reilly et al. (1999, Science 285:1926-1928) have discovered that the cleaved antithrombin has potent anti-angiogenic activity. An anti-angiogenic agent may be the anti-angiogenic form of antithrombin. An anti-angiogenic agent may be the 40 kDa and/or 29 kDa proteolytic fragment of fibronectin.

An anti-angiogenic agent may be a urokinase plasminogen activator (uPA) receptor antagonist or a dominant negative mutant of uPA (see, e.g., Crowley et al., 1993, Proc. Natl. Acad. Sci. USA 90:5021-5025). An antagonist may be a peptide antagonist or a fusion protein thereof (Goodson et al., 1994, Proc. Natl. Acad. Sci. USA 91:7129-7133). An antagonist may be a dominant negative soluble uPA receptor (Min et al., 1996, Cancer Res. 56:2428-2433). An antagonist may be the 16 kDa N-terminal fragment of prolactin, comprising approximately 120 amino acids, or a biologically active fragment thereof (the coding sequence for prolactin can be found in GenBank Accession No. NM_{—}000948). An anti-angiogenic agent may be the 7.8 kDa platelet factor-4 fragment. An anti-angiogenic agent may be a small peptide corresponding to the anti-angiogenic 13 amino acid fragment of platelet factor-4, the anti-angiogenic factor designated 13.40, the anti-angiogenic 22 amino acid peptide fragment of thrombospondin I, the anti-angiogenic 20 amino acid peptide fragment of SPARC, the small anti-angiogenic peptides of laminin, fibronectin, procollagen, or EGF, or small peptide antagonists of integrin αᵥβ₃ or the VEGF receptor. An anti-angiogenic agent may or may not be a TNF-α antagonist.

Nucleic acid molecules encoding proteins, polypeptides, or peptides with anti-angiogenic activity, or proteins, polypeptides or peptides with anti-angiogenic activity can be administered to a subject.

Proteins, polypeptides, or peptides that can be used as anti-angiogenic agents can be produced by any technique well-known in the art or described herein. Proteins, polypeptides or peptides with anti-angiogenic activity can be engineered so as to increase the in vivo half-life of such proteins, polypeptides, or peptides utilizing techniques well-known in the art or described herein. Anti-angiogenic agents that are commercially available can be used in the compositions and methods encompassed by the invention. An anti-angiogenic activity of an agent can be determined in vitro and/or in vivo by any technique well-known to one skilled in the art.

TNF-α Antagonists

Any TNF-α antagonist well-known to one of skill in the art may be used in the methods encompassed by the invention. Non-limiting examples of TNF-α antagonists include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that immunospecifically bind to TNF-α, nucleic acid molecules (e.g., antisense molecules or triple helices), organic molecules, inorganic molecules, and small molecules that blocks, reduces, inhibits or neutralizes a function, an activity and/or expression of TNF-α. In various embodiments, a TNF-α. antagonist reduces the function, activity and/or expression of TNF-α by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

Examples of antibodies that immunospecifically bind to TNF-α include, but are not limited to, infliximab (REMICADE®; Centacor), D2E7 (Abbott Laboratories/Knoll Pharmaceuticals Co., Mt. Olive, N.J.), CDP571 which is also known as HUMICADE™, adalimumab (Humira), certolizumab (Cimizia) and CDP-870 (both of Celltech/Pharmacia, Slough, U.K.), and TN3-19.12 (Williams et al., 1994, Proc. Natl. Acad. Sci. USA 91: 2762-2766; Thorbecke et al., 1992, Proc. Natl. Acad. Sci. USA 89:7375-7379). Antibodies that immunospecifically bind to TNF-α are also disclosed in the following U.S. Pat. Nos. 5,136,021; 5,147,638; 5,223,395; 5,231,024; 5,334,380; 5,360,716; 5,426,181; 5,436,154; 5,610,279; 5,644,034; 5,656,272; 5,658,746; 5,698,195; 5,736,138; 5,741,488; 5,808,029; 5,919,452; 5,958,412; 5,959,087; 5,968,741; 5,994,510; 6,036,978; 6,114,517; and 6,171,787. Examples of soluble TNF-α receptors include, but are not limited to, sTNF-R1 (Amgen), etanercept (ENBRELTM; Immunex) and its rat homolog RENBREL™, soluble inhibitors of TNF-α derived from TNFrI, TNFrII (Kohno et al., 1990, Proc. Natl. Acad. Sci. USA 87:8331-8335), and TNF-α Inh (Seckinger et al, 1990, Proc. Natl. Acad. Sci. USA 87:5188-5192).

A TNF-α antagonist may be a soluble TNF-α receptor. A TNF-α antagonist may be etanercept (ENBREL™; Immunex) or a fragment, derivative or analog thereof. A TNF-α antagonist may be an antibody that immunospecifically binds to TNF-α. A TNF-α antagonist may be infliximab (REMICADE; Centacor) a derivative, analog or antigen-binding fragment thereof.

Other TNF-α antagonists may be IL-10, which is known to block TNF-α production via interferon y-activated macrophages (Oswald et al. 1992, Proc. Natl. Acad. Sci. USA 89:8676-8680), TNFR-IgG (Ashkenazi et al., 1991, Proc. Natl. Acad. Sci. USA 88:10535-10539), the murine product TBP-1 (Serono/Yeda), the vaccine CytoTAb (Protherics), antisense molecule104838 (ISIS), the peptide RDP-58 (SangStat), thalidomide (Celgene), CDC-801 (Celgene), DPC-333 (Dupont), VX-745 (Vertex), AGIX-4207 (AtheroGenics), ITF-2357 (Italfarmaco), NPI-13021-31 (Nereus), SCIO-469 (Scios), TACE targeter (Immunix/AHP), CLX-120500 (Calyx), Thiazolopyrim (Dynavax), auranofin (Ridaura) (SmithKline Beecham Pharmaceuticals), quinacrine (mepacrine dichlorohydrate), tenidap (Enablex), Melanin (Large Scale Biological), and anti-p38 MAPK agents by Uriach.

Nucleic acid molecules encoding proteins, polypeptides, or peptides with TNF-α antagonist activity, or proteins, polypeptides, or peptides with TNF-α antagonist activity can be administered as a therapeutic agent. Further, nucleic acid molecules encoding derivatives, analogs, fragments or variants of proteins, polypeptides, or peptides with TNF-α antagonist activity, or derivatives, analogs, fragments or variants of proteins, polypeptides, or peptides with TNF-α antagonist activity can be administered as a therapeutic agent.

Proteins, polypeptides, or peptides that can be used as TNF-α antagonists can be produced by any technique well-known in the art or described herein. Proteins, polypeptides or peptides with TNF-α antagonist activity can be engineered so as to increase the in vivo half-life of such proteins, polypeptides, or peptides utilizing techniques well-known in the art or described herein. The antagonists can be commercially available and known to function as TNF-α antagonists.

Interferon α or Type I IFN Inhibitors

Any IFN-α antagonist or type I IFN inhibitor well-known to one of skill in the art may be used in the methods encompassed by the invention. Non-limiting examples of IFN-α antagonists or type I IFN antagonists include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that immunospecifically bind to type I IFN or IFN-α, nucleic acid molecules (e.g., antisense molecules or triple helices), organic molecules, inorganic molecules, and small molecules that blocks, reduces, inhibits or neutralizes a function, an activity and/or expression of type I IFN or IFN-α. A type I IFN or IFN-α. antagonist may reduce the function, activity and/or expression of type I IFN or IFN-α by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

An antibody specific for type I IFN or IFN-α, may be specific for any subtype(s) of type I IFN or IFNα. For instance, the antibody may be specific for any one of IFNα1, IFNα2, IFNα4, IFNα5, IFNα6, IFNα7, IFNα8, IFNα10, IFNα14, IFNα17, IFNα21, IFNβ, or IFNω. Alternatively, the antibody may be specific for any two, any three, any four, any five, any six, any seven, any eight, any nine, any ten, any eleven, any twelve type I IFN of IFNα subtypes. If the antibody is specific for more than one type I IFN subtype, the antibody may be specific for IFNα1, IFNα2, IFNα4, IFNα5, IFNα8, IFNα10, and IFNα21; or it may be specific for IFNα1, IFNα2, IFNα4, IFNα5, IFNα8, and IFNα10; or it may be specific for IFNα1, IFNα2, IFNα4, IFNα5, IFNα8, and IFNα21; or it may be specific for IFNα1, IFNα2, IFNα4, IFNα5, IFNα10, and IFNα21. Antibodies specific for type I IFN or IFNα include MEDI-545, any biologic or antibody other than MEDI-545, antibodies described in U.S. patent application Ser. No. 11/009,410 filed Dec. 10, 2004 and Ser. No. 11/157,494 filed Jun. 20, 2005, 9F3 and other IFN antibodies described in U.S. Pat. No. 7,087,726, NK-2 and YOK5/19 (WO 84/03105), LO-22 (U.S. Pat. No. 4,902,618), 144 BS (U.S. Pat. No. 4,885,166), and EBI-1, EBI-2, and EBI-3 (EP 119476).

An antibody specific for a type I IFN or IFN-α receptor may also be a type I IFN or IFN-α inhibitor. Type I IFN or IFN-α receptor antibodies may be any known in the art. These antibodies include, but are limited to, antibodies H2K6, H2K1, H3K65, and H3K1 in U.S. patent application publication 2005-0208041; antibodies 3F11, 4G5, 11E2, and 9D4 in U.S. patent application publication 2006-0029601; antibody 34F10 in U.S. Pat. No. 5,919,453; antibody 64G12 in U.S. Pat. No. 7,179,465; and antibodies 5A8, 2E8, 2H6, 4A7, and 2E1 in U.S. Pat. No. 6,713,609.

Anti-Inflammatory Therapies

Any anti-inflammatory therapeutic well-known to one of skill in the art may be used in the methods encompassed by the invention. Non-limiting examples of anti-inflammatory agents include non-steroidal anti-inflammatory drugs (NSAIDs), steroidal anti-inflammatory drugs, anticholinergics (e.g., atropine sulfate, atropine methylnitrate, and ipratropium bromide (ATROVENT™)), beta2-agonists (e.g., abuterol (VENTOLINM and PROVENTIL™), bitolterol (TORNALATE™), levalbuterol (XOPONEX™), metaproterenol (ALUPENT™), pirbuterol (MAXAIR™), terbutlaine (BRETHAIRE™ and BRETHINE™), albuterol (PROVENTIL™, REPETABS™, and VOLMAX™), formoterol (FORADIL AEROLIZER™), and salmeterol (SEREVENT™ and SEREVENT DISKUS™)), and methylxanthines (e.g., theophylline (UNIPHYL™, THEO-DUR™, SLO-BID™, AND TEHO-42™)). Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALLEVE™, NAPROSYN™), ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), corticosteroids (e.g., methylprednisolone (MEDROL™)), cortisone, hydrocortisone, prednisone (PREDNISONE™ and DELTASONE™), prednisolone (PRELONE™ and PEDIAPRED™), triamcinolone, azulfidine, and inhibitors of eicosanoids (e.g., prostaglandins, thromboxanes, and leukotrienes.

The anti-inflammatory agent may be an agent including adrenergic stimulants (e.g., catecholamines (e.g., epinephrine, isoproterenol, and isoetharine), resorcinols (e.g., metaproterenol, terbutaline, and fenoterol), and saligenins (e.g., salbutamol)), adrenocorticoids, blucocorticoids, corticosteroids (e.g., beclomethadonse, budesonide, flunisolide, fluticasone, triamcinolone, methylprednisolone, prednisolone, and prednisone), other steroids, beta2-agonists (e.g., albtuerol, bitolterol, fenoterol, isoetharine, metaproterenol, pirbuterol, salbutamol, terbutaline, formoterol, salmeterol, and albutamol terbutaline), anti-cholinergics (e.g., ipratropium bromide and oxitropium bromide), IL-4 antagonists (including antibodies), IL-5 antagonists (including antibodies), IL-13 antagonists (including antibodies), PDE4-inhibitor, NF-kB inhibitor, VLA-4 inhibitor, CpG, anti-CD23, selectin antagonists (TBC 1269), mast cell protease inhibitors (e.g., tryptase kinase inhibitors (e.g., GW-45, GW-58, and genisteine), phosphatidylinositide-3′ (PI3)-kinase inhibitors (e.g., calphostin C), and other kinase inhibitors (e.g., staurosporine) (see Temkin et al., 2002 J Immunol 169(5):2662-2669; Vosseller et al., 1997 Mol. Biol. Cell 8(5):909-922; and Nagai et al., 1995 Biochem Biophys Res Commun 208(2):576-581)), a C3 receptor antagonists (including antibodies), immunosuppressant agents (e.g., methotrexate and gold salts), mast cell modulators (e.g., cromolyn sodium (INTAL™) and nedocromil sodium (TILADE™)), and mucolytic agents (e.g., acetylcysteine)). In a specific embodiment, the anti-inflammatory agent is a leukotriene inhibitor (e.g., montelukast (SINGULAIR™), zafirlukast (ACCOLATE™), pranlukast (ONON™), or zileuton (ZYFLO™)

Anti-inflammatory agents may be antimediator drugs (e.g., antihistamines, corticosteroids, decongestants, sympathomimetic drugs (e.g., α-adrenergic and β-adrenergic drugs), TNX901 (Leung et al., 2003, N Engl J Med 348(11):986-993), IgE antagonists (e.g., antibodies rhuMAb-E25 omalizumab (see Finn et al., 2003 J Allergy Clin Immuno 111(2):278-284; Corren et al., 2003 J Allergy Clin Immuno 111(1):87-90; Busse and Neaville, 2001 Curr Opin Allergy Clin Immuno 1(1):105-108; and Tang and Powell, 2001, Eur J Pediatr 160(12): 696-704), K-12 and 6HD5 (see Miyajima et al., 2202 Int Arch Allergy Immuno 128(1):24-32), and mAB Hu-901 (see van Neerven et al., 2001 Int Arch Allergy Immuno 124(1-3):400), theophylline and its derivatives, glucocorticoids, and immunotherapies (e.g., repeated long-term injection of allergen, short course desensitization, and venom immunotherapy).

Anti-inflammatory therapies and their dosages, routes of administration, and recommended usage are known in the art and have been described in such literature as the Physician's Desk Reference (57th ed., 2003).

Anti-Viral Agents

Any anti-viral agent well-known to one of skill in the art may be used in the methods encompassed by the invention. Non-limiting examples of anti-viral agents include proteins, polypeptides, peptides, fusion proteins antibodies, nucleic acid molecules, organic molecules, inorganic molecules, and small molecules that inhibit and/or reduce the attachment of a virus to its receptor, the internalization of a virus into a cell, the replication of a virus, or release of virus from a cell. In particular, anti-viral agents include, but are not limited to, nucleoside analogs (e.g., zidovudine, acyclovir, gangcyclovir, vidarabine, idoxuridine, trifluridine, and ribavirin), foscarnet, amantadine, rimantadine, saquinavir, indinavir, ritonavir, alpha-interferons and other interferons, and AZT.

The anti-viral agent may be an immunomodulatory agent that is immunospecific for a viral antigen. For example, such anti-viral agents include PRO542 (Progenics); Ostavir (Protein Design Labs, Inc., CA); Protovir (Protein Design Labs, Inc., CA); and palivizumab (SYNAGIS®; MedImmune, Inc.).

Anti-viral agents may include, but are not limited to, nucleoside analogs, such as zidovudine, acyclovir, gangcyclovir, vidarabine, idoxuridine, trifluridine, and ribavirin, as well as foscarnet, amantadine, rimantadine, saquinavir, indinavir, ritonavir, and the alpha-interferons. See U.S. Prov. Patent App. No. 60/398,475 filed Jul. 25, 2002, entitled “Methods of Treating and Preventing RSV, HMPV, and PIV Using Anti-RSV, Anti-HMPV, and Anti-PIV Antibodies,” and U.S. patent application Ser. No. 10/371,122 filed Feb. 21, 2003, which are incorporated herein by reference in its entirety.

Anti-viral therapies and their dosages, routes of administration and recommended usage are known in the art and have been described in such literature as the Physician ‘s Desk Reference (56^{th} ed., 2002). Additional information on respiratory viral infections is available in Cecil Textbook of Medicine (18th ed., 1988).

Anti-Bacterial Agents

Any anti-bacterial agent well-known to one of skill in the art may be used in the methods encompassed by the invention. Non-limiting examples of anti-bacterial agents include proteins, polypeptides, peptides, fusion proteins, antibodies, nucleic acid molecules, organic molecules, inorganic molecules, and small molecules that inhibit or reduce a bacterial infection, inhibit or reduce the replication of bacteria, or inhibit or reduce the spread of bacteria to other subjects. Examples of anti-bacterial agents include, but are not limited to, penicillin, cephalosporin, imipenem, axtreonam, vancomycin, cycloserine, bacitracin, chloramphenicol, erythromycin, clindamycin, tetracycline, streptomycin, tobramycin, gentamicin, amikacin, kanamycin, neomycin, spectinomycin, trimethoprim, norfloxacin, rifampin, polymyxin, amphotericin B, nystatin, ketocanazole, isoniazid, metronidazole, and pentamidine.

The anti-bacterial agent may be a tetracycline, erythromycin, or spectinomycin. The anti-bacterial agent is preferably penicillin, first second, or third generation cephalosporin (e.g., cefaclor, cefadroxil, cephalexin, or cephazolin), erythomycin, clindamycin, an aminoglycoside (e.g., gentamicin, tobramycin, or amikacine), or a monolactam (e.g., aztreonam). The anti-bacterial agent may be rifampcin, isonaizid, pyranzinamide, ethambutol, or streptomycin.

Anti-bacterial therapies and their dosages, routes of administration and recommended usage are known in the art and have been described in such literature as the Physician ‘s Desk Reference (56^{th} ed., 2002). Additional information on anti-bacterial therapies is available in Cecil Textbook of Medicine (18th ed., 1988).

Anti-Fungal Agents

Any anti-fungal agent well-known to one of skill in the art may be used in the methods encompassed by the invention. Non-limiting examples of anti-fungal agents include proteins, polypeptides, peptides, fusion proteins, antibodies, nucleic acid molecules, organic molecules, inorganic molecules, and small molecules that inhibit and/or reduce fungal infection, inhibit and/or reduce the replication of fungi, or inhibit and/or reduce the spread of fungi to other subjects. Specific examples of anti-fungal agents include, but are not limited to, azole drugs (e.g., miconazole, ketoconazole (NIZORAL®), caspofungin acetate (CANCIDAS®), imidazole, triazoles (e.g., fluconazole (DIFLUCAN®)), and itraconazole (SPORANOX®)), polyene (e.g., nystatin, amphotericin B (FUNGIZONE®), amphotericin B lipid complex (“ABLC”)(ABELCET®), amphotericin B colloidal dispersion (“ABCD”)(AMPHOTEC®), liposomal amphotericin B (AMBISONE®)), potassium iodide (KI), pyrimidine (e.g., flucytosine (ANCOBON®)), and voriconazole (VFEND®).

Anti-fungal therapies and their dosages, routes of administration, and recommended usage are known in the art and have been described in such literature as Dodds et al., 2000 Pharmacotherapy 20(11) 1335-1355, the Physician's Desk Reference (57th ed., 2003) and the Merck Manual of Diagnosis and Therapy (17th ed., 1999).

Therapeutic Agents, Inflammatory Bowel Disease

If the subject has been identified as a candidate for treatment of inflammatory bowel disease, e.g., a subject in which it may be desirable to prevent, treat, manage, and/or ameliorate inflammatory bowel disease or one or more symptoms thereof, the subject may be administered an inhibitor of IL-9 and a second therapeutic agent. The second therapeutic agent may be any one or more an antidiarrheal (e.g., loperamide 2-4 mg up to 4 times a day, diphenoxylate with atropine 1 tablet up to 4 times a day, tincture of opium 8-15 drops up to 4 times a day, cholestyramine 2-4 g or colestipol 5 g once or twice daily), antispasmodics (e.g., propantheline 15 mg, dicyclomine 10-20 mg, or hyoscyamine 0.125 mg given before meals), a 5-aminosalicylic acid agent (e.g., sulfasalazine 1.5-2 g twice daily, mesalamine (ASACOL®) and its slow release form (PENTASA®), especially at high dosages, e.g., PENTASA® 1 g four times daily and ASACOL® 0.8-1.2 g four times daily), a corticosteroid (e.g., prednisone, budesonide, hydrocortisone), an immunomodulatory drug (e.g., azathioprine (1-2 mg/kg), mercaptopurine (50-100 mg), cyclosporine, and methotrexate), an antibiotic (e.g., metronidazole, ciprofloxacin), a TNF inhibitor (e.g., inflixmab (REMICADE®); adalimumab (Humira, Abbot); certolizumab), an immunosuppressive agent (e.g., tacrolimus, mycophenolate mofetil, azathioprine, 6-mercaptopurine, and thalidomide), an anti-inflammatory cytokine (e.g., IL-10 and IL-11), a nutritional therapy, an enteral therapy with elemental diets (e.g., Vivonex for 4 weeks), or a total parenteral nutrition. The therapeutic agent may alternatively be an antibody specific for any subtype(s) of type I IFN or IFNα. For instance, the antibody may be specific for any one of IFNα1, IFNα2, IFNα4, IFNα5, IFNα6, IFNα7, IFNα8, IFNα10, IFNα14, IFNα17, IFNα21, IFNβ, or IFNω. Alternatively, the antibody may be specific for any two, any three, any four, any five, any six, any seven, any eight, any nine, any ten, any eleven, any twelve type I IFN of IFNα subtypes. If the antibody is specific for more than one type I IFN subtype, the antibody may be specific for IFNα1, IFNα2, IFNα4, IFNα5, IFNα8, IFNα10, and IFNα21; or it may be specific for IFNα1, IFNα2, IFNα4, IFNα5, IFNα8, and IFNα10; or it may be specific for IFNα1, IFNα2, IFNα4, IFNα5, IFNα8, and IFNα21; or it may be specific for IFNα1, IFNα2, IFNα4, IFNα5, IFNα10, and IFNα21. Antibodies specific for type I IFN or IFNα include MEDI-545, any biologic or antibody other than MEDI-545, antibodies described in U.S. patent application Ser. No. 11/009,410 filed Dec. 10, 2004 and Ser. No. 11/157,494 filed Jun. 20, 2005, 9F3 and other IFN antibodies described in U.S. Pat. No. 7,087,726, NK-2 and YOK5/19 (WO 84/03105), LO-22 (U.S. Pat. No. 4,902,618), 144 BS (U.S. Pat. No. 4,885,166), and EBI-1, EBI-2, and EBI-3 (EP 119476).

Therapeutic Agent, COPD

If the subject has been identified as a candidate for treatment of COPD, e.g., a subject in which it may be desirable to prevent, treat, manage, and/or ameliorate COPD or one or more symptoms thereof, the subject may be administered an inhibitor of IL-9 and a second therapeutic agent. The second therapeutic agent may be any one or more of a bronchodilator (e.g. short-acting β2-adrenergic agonist (e.g., albuterol, pirbuterol, terbutaline, and metaproterenol), a long-acting β₂-adrenergic agonist (e.g., oral sustained-release albuterol and inhaled salmeterol), an anticholinergic (e.g., ipratropium bromide), or theophylline or its derivatives (therapeutic range for theophylline is preferably 10-201 g/mL)), a glucocorticoid, exogenous α₁AT (e.g., α₁AT derived from pooled human plasma administered intravenously in a weekly dose of 60 mg/kg), oxygen, lung transplantation, lung volume reduction surgery, endotracheal intubation, ventilation support, yearly influenza vaccine and pneumococcal vaccination with 23-valent polysaccharide, exercise, or smoking cessation.

Therapeutic Agent Which is Not an Inhibitor of IL-9-Pulmonary Fibrosis

If the subject has been identified as a candidate for treatment of pulmonary fibrosis, e.g., a subject in which it may be desirable to prevent, treat, manage, and/or ameliorate pulmonary fibrosis or one or more symptoms thereof, the subject may be administered an inhibitor of IL-9 and a second therapeutic agent. The second therapeutic agent may be any one or more of oxygen, corticosteroids (e.g., daily administration of prednisone beginning at 1-1.5 mg/kg/d (up to 100 mg/d) for six weeks and tapering slowly over 3-6 months to a minimum maintenance dose of 0.25 mg/kg/d), a cytotoxic drug (e.g., cyclophosphamide at 100-120 mg orally once daily and azathioprine at 3 mg/kg up to 200 mg orally once daily), a bronchodilator (e.g., short- and long-acting β₂-adrenergic agonists, anticholinergics, and theophylline and its derivatives), or an antihistamine (e.g., diphenhydramine and doxylamine).

Administration

The amount of an inhibitor of IL-9 to be administered can be determined by standard clinical methods. The frequency and dosage will vary also according to factors specific for each patient depending on the specific therapies (e.g., the specific therapeutic or prophylactic agent or agents) administered, the severity of the disorder, disease, or condition, the route of administration, as well as age, body, weight, response, and the past medical history of the patient. For example, the dosage of a prophylactic or therapeutic agent or a composition of the invention which will be effective in a treatment, or one or more symptoms thereof, can be determined by administering the composition to an animal model such as, e.g., the animal models disclosed herein or known in to those skilled in the art. In addition, in vitro assays may optionally be employed to help identify optimal dosage ranges. Suitable regimens can be selected by one skilled in the art by considering such factors and by following, for example, dosages are reported in literature and recommended in the Physician ‘s Desk Reference (57th ed., 2003).

Doses of a small molecule may include milligram or microgram amounts per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

For antibodies, proteins, polypeptides, peptides and fusion proteins encompassed by the invention, the dosage administered to a patient may be 0.0001 mg/kg to 100 mg/kg of the patient's body weight. The dosage administered to a patient may be between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration may be possible. Further, the dosage and frequency of administration of antibodies of the invention or fragments thereof may be reduced by enhancing uptake and tissue penetration of the antibodies by modifications such as, for example, lipidation.

The dosage administered to a patient may be calculated using the patient's weight in kilograms (kg) multiplied by the dose to be administered in mg/kg. The required volume (in mL) to be given may then be determined by taking the mg dose required divided by the concentration of the antibody or fragment thereof in the formulations (100 mg/mL). The final calculated required volume may be obtained by pooling the contents of as many vials as are necessary into syringe(s) to administer the drug.

The dosage of antibodies whether in a composition as a stand alone therapy or in a combination therapy may be 150 μg/kg or less, preferably 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight. The dosage of antibodies in a composition may be in a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 m g, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

The one or more doses of an effective amount of an IL-9 inhibitor (alone or in combination with a second therapeutic agent) may prevent at least 20% to 25%, at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% of endogenous IL-9 from binding to its receptor. The one or more doses may reduce and/or inhibit mast cell degranulation at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, at least 80 to 85%, at least 85% to 90%, at least 90% to 95%, or at least 95% to 98% relative to a control such as PBS in an in vitro and/or in vivo assay well-known in the art. The one or more doses of an effective amount of an IL-9 inhibitor (alone or in combination with a second therapeutic agent) may reduce and/or inhibit mast cell activation at least 20% to 25%, at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, at least 80 to 85%, at least 85% to 90%, at least 90% to 95%, or at least 95% to 98% relative to a control such as PBS in an in vitro and/or in vivo assay well-known in the art. The one or more doses of an effective amount of an IL-9 inhibitor (alone or in combination with a second therapeutic agent) may reduce and/or inhibit mast cell proliferation at least 20% to 25%, at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, at least 80 to 85%, at least 85% to 90%, at least 90% to 95%, or at least 95% to 98% relative to a control such as PBS in an in vitro and/or in vivo assay well-known in the art. The one or more doses of an effective amount of an IL-9 inhibitor (alone or in combination with a second therapeutic agent) may reduce and/or inhibit mast cell infiltration at least 20% to 25%, at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, at least 80 to 85%, at least 85% to 90%, at least 90% to 95%, or at least 95% to 98% relative to a control such as PBS in an in vitro and/or in vivo assay well-known in the art.

If the IL-9 inhibitor is an antibody specific for IL-9, the dose may achieve a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml.

If the IL-9 inhibitor is an antibody specific for IL-9, the dose may be at least 10 μg, at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, or at least 120 μg. The antibody specific for IL-9 may be administered every 3 days, once every 4 days, once every 5 days, once every 6 days, once every 7 days, once every 8 days, once every 10 days, once every two weeks, once every three weeks, or once a month.

If the IL-9 inhibitor is an antibody specific for IL-9, the dose may be at least 10 μg (at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg). The dose may be administered to a subject one or more times such that the plasma level of the antibody in the subject is less than 0.1 μg/ml, less than 0.25 μg/ml, less than 0.5 μg/ml, less than 0.75 μg/ml, or less than 1 μg/ml. The dose may be administered 1, 2, 3, 4, 5, 6, 7, 8, 9, 10, 11, or 12 times. The doses of the antibody may be repeated at intervals of at least 1 day, 2 days, 3 days, 5 days, 10 days, 15 days, 30 days, 45 days, 2 months, 75 days, 3 months, or at least 6 months. If the IL-9 inhibitor is not an antibody (e.g., prophylactic or therapeutic agent) the doses may be repeated and may be separated by at least at least 1 day, 2 days, 3 days, 5 days, 10 days, 15 days, 30 days, 45 days, 2 months, 75 days, 3 months, or at least 6 months.

Therapeutic agents (e.g., prophylactic or therapeutic agents), other than antibodies that specifically bind IL-9, can be administered in combination with the antibodies that specifically bind IL-9. The recommended dosages of these therapeutic agents can be obtained from any reference in the art including, but not limited to, Hardman et al., eds., 2001, Goodman & Gilman's The Pharmacological Basis Of Basis Of Therapeutics, 10th ed., Mc-Graw-Hill, New York; Physician's Desk Reference (PDR) 57th ed., 2003, Medical Economics Co., Inc., Montvale, N.J.

An antibody specific for IL-9 and a therapeutic agent that is not an antibody specific for IL-9 (e.g., prophylactic or therapeutic agents) can be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The antibody specific for IL-9 and the therapeutic agent that is not an antibody specific for IL-9 can be administered at the same time in one, or as separate compositions.

An antibody specific for IL-9 and a therapeutic agent that is not an antibody specific for IL-9 (e.g., prophylactic or therapeutic agents) can be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

### Pharmaceutical Compositions

The inhibitors of IL-9 employed in the methods encompassed by the invention may comprise one or more peptides, polypeptides, or proteins comprising a fragment of an antibody of the invention that immunospecifically binds IL-9. Alternatively, the inhibitors of IL-9 employed in the methods encompassed by the invention may comprise compounds other than a peptide, polypeptide, or protein comprising a fragment of an antibody specific for IL-9, e.g., a 3-aminosteroid compound.

A composition employed by the methods encompassed by the invention may comprise an antibody specific for IL-9 and one or more immunomodulatory agents. A composition employed by the methods encompassed by the invention may comprise an antibody specific for IL-9 and one or more mast cell modulators. A composition employed by the methods encompassed by the invention may comprise an antibody specific for IL-9 and one or more anti-angiogenic agents. A composition employed by the methods encompassed by the invention may comprise an antibody specific for IL-9 and one or more anti-inflammatory agents. A composition employed by the methods encompassed by the invention may comprise an antibody specific for IL-9 and one or more anti-viral agents. A composition employed by the methods encompassed by the invention may comprise and antibody specific for IL-9 an one or more anti-bacterial agents. A composition employed by the methods encompassed by the invention may comprise an antibody specific for IL-9 and one or more anti-fungal agents. A composition employed by the methods encompassed by the invention may comprise an antibody specific for IL-9 and any combination of one, two, three, or more of each of the following prophylactic or therapeutic agents: an immunomodulatory agent, a mast cell modulator, an anti-angiogenic agent, an anti-cancer agent other than an immunomodulatory agent or anti-angiogenic agent, an anti-inflammatory agent, an anti-viral agent, an anti-bacterial agent, an anti-fungal agent.

A composition employed by the methods encompassed by the invention may comprise an antibody specific for IL-9 and one or more TNF-α antagonists (e.g., ENBREL™ and/or REMICADE®). A composition employed by the methods encompassed by the invention may comprise an antibody specific for IL-9 and one or more integrin αᵥβ₃ antagonists. A composition employed by the methods encompassed by the invention may comprise an antibody specific for IL-9 and VITAXIN™, siplizumab, palivizumab, an EphA2 inhibitor, or any combination thereof. In addition to prophylactic or therapeutic agents, the compositions of the invention may also comprise a carrier.

A composition employed by the methods encompassed by the invention may comprise a small molecule inhibitor or IL-9 and one or more immunomodulatory agents. A composition employed by the methods encompassed by the invention may comprise a small molecule inhibitor or IL-9 and one or more mast cell modulators. A composition employed by the methods encompassed by the invention may comprise a small molecule inhibitor of IL-9 and one or more anti-angiogenic agents. A composition employed by the methods encompassed by the invention may comprise a small molecule inhibitor of IL-9 and one or more anti-inflammatory agents. A composition employed by the methods encompassed by the invention may comprise a small molecule inhibitor of IL-9 and one or more anti-viral agents. A composition employed by the methods encompassed by the invention may comprise and antibody specific for IL-9 an one or more anti-bacterial agents. A composition employed by the methods encompassed by the invention may comprise a small molecule inhibitor of IL-9 and one or more anti-fungal agents. A composition employed by the methods encompassed by the invention may comprise a small molecule of IL-9 and any combination of one, two, three, or more of each of the following prophylactic or therapeutic agents: an immunomodulatory agent, a mast cell modulator, an anti-angiogenic agent, an anti-cancer agent other than an immunomodulatory agent or anti-angiogenic agent, an anti-inflammatory agent, an anti-viral agent, an anti-bacterial agent, an anti-fungal agent. The small molecule inhibitor may be a 3-aminosteroid compound as disclosed in U.S. Pat. No. 7,074,778. The small molecule inhibitor may be a soluble IL-9 receptor. See, e.g., U.S. Pat. No. 6,602,850.

The inhibitors of IL-9 may be bulk drug compositions useful in the manufacture of pharmaceutical compositions (e.g., compositions that are suitable for administration to a subject or patient) which can be used in the preparation of unit dosage forms (See U.S patent application publication 2005/0260204 entitled “Anti-IL-9 Antibody Formulations and Uses Thereof” for forms which may be prepared for IL-9 specific antibodies). A pharmaceutical composition may comprise an inhibitor of IL-9 and a pharmaceutically acceptable carrier. The pharmaceutical compositions may be formulated to be suitable for the route of administration to a subject. A phamaceutical composition comprising an antibody specific for IL-9 may be formulated in single dose vials as a sterile liquid that contains 10 mM histidine buffer at pH 6.0 and 150 mM sodium chloride. Each 1.0 mL of solution contains 100 mg of protein, 1.6 mg of histidine and 8.9 mg of sodium chloride in water for optimal stability and solubility.

“Pharmaceutically acceptable” is approved by a regulatory agency of the Federal or a state government or listed in the U.S. Pharmacopeia or other generally recognized pharmacopeia for use in animals, and more particularly in humans. A carrier refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water may be the carrier of a pharmaceutical composition administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Ingredients of compositions comprising an inhibitor of IL-9 may be supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Compositions comprising an inhibitor of IL-9 can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Various delivery systems are known in the art and can be used to administer a prophylactic or therapeutic agent or composition comprising an inhibitor of IL-9, e.g., encapsulation in liposomes, microparticles, microcapsules, recombinant cells capable of expressing an IL-9 antibody or antibody fragment, receptor-mediated endocytosis (see, e.g., Wu and Wu, J. Biol. Chem. 262:4429-4432 (1987)), construction of a nucleic acid as part of a retroviral or other vector, etc.

Methods of administering an inhibitor of IL-9 (e.g., prophylactic or therapeutic agent) include, but are not limited to, parenteral administration (e.g., intradermal, intramuscular, intraperitoneal, intravenous and subcutaneous), epidurala administration, intratumoral administration, and mucosal adminsitration (e.g., intranasal and oral routes). In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety. An inhibitor of IL-9 may be administered using Alkermes AIRTM pulmonary drug delivery technology (Alkermes, Inc., Cambridge, Mass.). Inhibitors of IL-9 and/or other therapeutic agents may be administered intramuscularly, intravenously, intratumorally, orally, intranasally, pulmonary, or subcutaneously. Inhibitors of IL-9 and/or other therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

Inhibitors of IL-9 may be administered locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices (e.g., Tissuel®), or collagen matrices.

An inhibitor of IL-9 can be delivered in a controlled release or sustained release system. For example, a pump may be used to achieve controlled or sustained release (see Langer, supra; Sefton, 1987, CRC Crit. Ref Biomed. Eng. 14:20; Buchwald et al., 1980, Surgery 88:507; Saudek et al., 1989, N. Engl. J. Med. 321:574). Alternatively, polymeric materials can be used to achieve controlled or sustained release of the therapies of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer may be used in a sustained release formulation is inert, free of leachable impurities, stable on storage, sterile, and biodegradable. A controlled or sustained release system may be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising an inhibitor of IL-9 and/or one or more therapeutic agents. See, e.g., U.S. Pat. No. 4,526,938, PCT publication WO 91/05548, PCT publication WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760.

If the inhibitor of IL-9 or other therapeutic is a nucleic acid, the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see, e.g., Joliot et al., 1991, Proc. Natl. Acad. Sci. USA 88:1864-1868). Alternatively, a nucleic acid can be introduced intracellularly and incorporated within host cell DNA for expression by homologous recombination.

A pharmaceutical composition comprising an inhibitor of IL-9 may be formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. The composition may be formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

If the compositions comprising an inhibitor of IL-9 are to be administered topically, the compositions can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity preferably greater than water may be employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the inhibitor of IL-9 is administered intranasally, it can be formulated in an aerosol form, spray, mist or in the form of drops. Such formulations can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

If the inhibitor of IL-9 is administered orally, it may be formulated in the form of tablets, capsules, cachets, gelcaps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

If the inhibitor of IL-9 is administered pulmonarily, e.g., by use of an inhaler or nebulizer, it may be formulated with an aerosolizing agent. See, e.g., U.S. Pat. Nos. 6,019,968, 5,985, 320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903. The inhibitor of IL-9 may be administered using Alkermes AIRTM pulmonary drug delivery technology (Alkermes, Inc., Cambridge, Mass.).

If the inhibitor of IL-9 is administered in a formulation for parenteral administration by injection (e.g., by bolus injection or continuous infusion) it may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

The inhibitor of IL-9 may be administered as a depot preparation. Such long acting formulations may be administered by implantation (e.g., subcutaneously or intramuscularly) or by intramuscular injection. Thus, for example, the inhibitor of IL-9 may be formulated with suitable polymeric or hydrophobic materials (e.g., as an emulsion in an acceptable oil) or ion exchange resins, or as sparingly soluble derivatives (e.g., as a sparingly soluble salt).

The inhibitor of IL-9 may be formulated for administration as a neutral or salt form. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Ingredients of compositions comprising an inhibitor of IL-9 may be supplied either separately or mixed together in unit dosage form, for example, as a dry lyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration.

An inhibitor of IL-9 to be administered may be packaged in a hermetically sealed container such as an ampoule or sachette indicating the quantity of the agent. The inhibitor of IL-9 may be supplied as a dry sterilized lyophilized powder or water free concentrate in a hermetically sealed container and can be reconstituted (e.g., with water or saline) to the appropriate concentration for administration to a subject. The inhibitor of IL-9 may be supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. Alternatively, an inhibitor of IL-9 may be supplied in liquid form in a hermetically sealed container indicating the quantity and concentration of the agent. The liquid form may be supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml.

### Screening Inhibitors of IL-9 as Therapeutics

Inhibitors of IL-9 can be tested in vitro and/or in vivo for their ability to modulate the biological activity of immune cells (e.g., T cells, neutrophils, and mast cells), endothelial cells, and epithelial cells. The ability of inhibitors of IL-9 to modulate the biological activity of immune cells (e.g., T cells, B cells, mast cells, macrophages, neutrophils, and eosinophils), endothelial cells, and epithelial cells can be assessed by: detecting the expression of antigens (e.g., activation of genes by IL-9, such as, but not limited to, mucin genes (e.g., MUC2, MUC5AC, MUC5B, and MUC6) and genes involved in lymphocyte activation (e.g., Lgamma-6A/E)); detecting the proliferation of immune cells, endothelia cells and/or epithelial cells; detecting the activation of signaling molecules (e.g., the phosphorylation of Stat2, the phosphorylation of JAK3, or the phosphorylation of the IL-9R); detecting the effector function of immune cells (e.g., T cells, B cells, mast cells, macrophages, neutrophils, and eosinophils), endothelial cells, and/or epithelial cells; or detecting the differentiation of immune cells, endothelial cells, and/or epithelial cells. Techniques known to those of skill in the art can be used for measuring these activities. For example, cellular proliferation can be assayed by 3H-thymidine incorporation assays and trypan blue cell counts. Antigen expression can be assayed, for example, by immunoassays including, but are not limited to, competitive and non-competitive assay systems using techniques such as western blots, immunohistochemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and FACS analysis. The activation of signaling molecules can be assayed, for example, by kinase assays and electrophoretic shift assays (EMSAs). Mast cell degranulation can be assayed, for example by measuring serotonin (5-HT) release or histamine release with high-performance liquid chromatogoraphy (see, e.g., Taylor et al. 1995 Immunology 86(3): 427-433 and Kurosawa et al., 1998 Clin Exp Allergy 28(8): 1007-1012).

Inhibitors of IL-9 can be tested in vitro and then in vivo for the desired therapeutic or prophylactic activity prior to use in humans. For example, assays which can be used to determine whether administration of a specific pharmaceutical composition is indicated include cell culture assays in which a patient tissue sample is grown in culture and exposed to, or otherwise contacted with, a pharmaceutical composition, and the effect of such composition upon the tissue sample is observed. The tissue sample can be obtained by biopsy from the patient. This test allows the identification of the therapeutically most effective therapy (e.g., prophylactic or therapeutic agent) for each individual patient. In various specific embodiments, in vitro assays can be carried out with representative cells of cell types involved a fibrotic or inflammatory disease or disorder) to determine if a pharmaceutical composition of the invention has a desired effect upon such cell types.

The effect of an IL-9 inhibitor on peripheral blood lymphocyte counts can be monitored/assessed using standard techniques known to one of skill in the art. Peripheral blood lymphocytes counts in a subject can be determined by, e.g., obtaining a sample of peripheral blood from said subject, separating the lymphocytes from other components of peripheral blood such as plasma using, e.g., Ficoll-Hypaque (Pharmacia) gradient centrifugation, and counting the lymphocytes using trypan blue. Peripheral blood T-cell counts in subject can be determined by, e.g., separating the lymphocytes from other components of peripheral blood such as plasma using, e.g., a use of Ficoll-Hypaque (Pharmacia) gradient centrifugation, labeling the T-cells with an antibody directed to a T-cell antigen which is conjugated to FITC or phycoerythrin, and measuring the number of T-cells by FACS.

Inhibitors of IL-9 can be tested in suitable animal model systems prior to use in humans. Such animal model systems include, but are not limited to, rats, mice, chicken, cows, monkeys, pigs, dogs, rabbits, etc. Any animal system well-known in the art may be used. Several aspects of the procedure may vary; said aspects include, but are not limited to, the temporal regime of administering the therapies (e.g., prophylactic and/or therapeutic agents), whether such therapies are administered separately or as an admixture, and the frequency of administration of the therapies.

The anti-inflammatory activity of an inhibitor of IL-9 can be determined by using various experimental animal models of inflammatory arthritis known in the art and described in Crofford L. J. and Wilder R. L., “Arthritis and Autoimmunity in Animals,” in Arthritis and Allied Conditions: A Textbook of Rheumatology, McCarty (eds.), Chapter 30 (Lee and Febiger, 1993). Experimental and spontaneous animal models of inflammatory arthritis and autoimmune rheumatic diseases can also be used to assess the anti-inflammatory activity of the inhibitors of IL-9.

The anti-inflammatory activity of an inhibitor of IL-9 can also be assessed by measuring the inhibition of carrageenan-induced paw edema in the rat, using a modification of the method described in Winter C. A. et al., “Carrageenan-Induced Edema in Hind Paw of the Rat as an Assay for Anti-inflammatory Drugs” Proc. Soc. Exp. Biol Med. 111, 544-547, (1962). This assay has been used as a primary in vivo screen for the anti-inflammatory activity of most NSAIDs, and is considered predictive of human efficacy. The anti-inflammatory activity of the test therapies (e.g., prophylactic or therapeutic agents) is expressed as the percent inhibition of the increase in hind paw weight of the test group relative to the vehicle dosed control group.

An experimental animal model that can be used is an adjuvant-induced arthritis rat model. Body weight can be measured relative to a control group to determine the anti-inflammatory activity of an inhibitor of IL-9.

Efficacy in preventing or treating an inflammatory disorder may be demonstrated, e.g., by detecting the ability of inhibitor of IL-9 to reduce one or more symptoms of the inflammatory disorder, to decrease T cell activation, to decrease T cell proliferation, to modulate one or more cytokine profiles, to reduce cytokine production, to reduce inflammation of a joint, organ or tissue or to improve quality of life.

Changes in inflammatory disease activity may also be assessed through tender and swollen joint counts, patient and physician global scores for pain and disease activity, and the ESR/CRP. Progression of structural joint damage may be assessed by quantitative scoring of X-rays of hands, wrists, and feet (Sharp method). Changes in functional status in humans with inflammatory disorders may be evaluated using the Health Assessment Questionnaire (HAQ), and quality of life changes are assessed with the SF.

Experimental mouse models are available to those of skill in the art for assessing efficacy, therapeutic dose and safety of inhibitors of IL-9 in inflammatory bowel disease. See, e.g., Pizarro et al. Trends Mol. Med. 9 (2003):218-222 & Neurath et al. J. Exp. Med. 182 (1995):1281-1290).

The toxicity and/or efficacy of the prophylactic and/or therapeutic protocols employing an inhibitor of IL-9 can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. While therapies that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED50 with little or no toxicity. The dosage may vary within this range depending upon the dosage form employed and the route of administration utilized. For any therapy used in the methods encompassed by the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Further, any assays known to those skilled in the art can be used to evaluate the prophylactic and/or therapeutic utility of inhibitor of IL-9 for a fibrotic or inflammatory disease or disorder.

### Recombinant Expression of Antibodies

If the inhibitor of IL-9 is an antibody immunospecific for IL-9, it may be produced recombinantly. Recombinant expression of an antibody that immunospecifically binds IL-9 requires construction of an expression vector(s) containing a polynucleotide that encodes the antibody or a portion thereof. Once a polynucleotide(s) encoding the antibody molecule has been obtained, a vector for the production of the antibody molecule may be produced by recombinant DNA technology using techniques well-known in the art. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. Vectors including the nucleotide sequence encoding the constant region of the antibody molecule (see, e.g., International Publication No. WO 86/05807; International Publication No. WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. A variety of host-expression vector systems may be utilized to express an antibody immunospecific for IL-9. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5 K promoter). Bacterial cells, such as E. coli, and eukaryotic cells can be used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). The expression of nucleotide sequences encoding antibodies immunospecific for IL-9 can be regulated by a constitutive promoter, inducible promoter or tissue specific promoter.

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) can be used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, WI 38, BT483, Hs578T, HTB2, BT2O and T47D, NS0 (a murine myeloma cell line that does not endogenously produce any immunoglobulin chains), CRL7O3O and HsS78Bst cells.

The expression levels of an antibody that immunospecifically binds IL-9 can be increased by vector amplification (for a review, see Bebbington and Hentschel). The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody that immunospecifically binds IL-9 has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins.

### Methods of Producing Polypeptides

If the inhibitor of IL-9 is a polypeptide, peptide, protein, or fusion protein that is not an antibody, it can be produced by standard recombinant DNA techniques or by protein synthetic techniques, e.g., by use of a peptide synthesizer. For example, a nucleic acid molecule encoding a polypeptide, peptide, protein, or fusion protein that inhibits IL-9 can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al., eds., John Wiley & Sons, 1992).

Nucleotide sequences encoding a polypeptide, peptide, protein, or fusion protein inhibitor of IL-9, e.g., a soluble IL-9 receptor polypeptide, may be obtained from any information available to those of skill in the art (i.e., from Genbank, the literature, or by routine cloning). The nucleotide sequence coding for a polypeptide, peptide, protein, and fusion protein inhibitor of IL-9 can be inserted into an appropriate expression vector, i.e., a vector which contains the necessary elements for the transcription and translation of the inserted protein-coding sequence. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

The expression of a polypeptide, peptide, protein, or fusion protein inhibitor of IL-9 may be controlled by any promoter or enhancer element known in the art. Promoters which may be used to control the expression of the gene encoding fusion protein include, but are not limited to, the SV40 early promoter region (Bemoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the tetracycline (Tet) promoter (Gossen et al., 1995, Proc. Nat. Acad. Sci. USA 89:5547-5551); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25; see also “Useful proteins from recombinant bacteria” in Scientific American, 1980, 242:74-94); plant expression vectors comprising the nopaline synthetase promoter region (Herrera-Estrella et al., Nature 303:209-213) or the cauliflower mosaic virus ³⁵S RNA promoter (Gardner, et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); neuronal-specific enolase (NSE) which is active in neuronal cells (Morelli et al., 1999, Gen. Virol. 80:571-83); brain-derived neurotrophic factor (BDNF) gene control region which is active in neuronal cells (Tabuchi et al., 1998, Biochem. Biophysic. Res. Corn. 253:818-823); glial fibrillary acidic protein (GFAP) promoter which is active in astrocytes (Gomes et al., 1999, Braz J Med Biol Res 32(5):619-631; Morelli et al., 1999, Gen. Virol. 80:571-83) and gonadotropic releasing hormone gene control region which is active in the hypothalamus (Mason et al., 1986, Science 234:1372-1378).

The expression of a polypeptide, peptide, protein, or fusion protein inhibitor of IL-9 may be regulated by a constitutive promoter. The expression of a polypeptide, peptide, protein, or a fusion protein inhibitor of IL-9 may be regulated by an inducible promoter. The expression of a polypeptide, peptide, protein, or a fusion protein inhibitor of IL-9 may be regulated by a tissue-specific promoter.

In mammalian host cells, a number of viral-based expression systems may be utilized. In cases where an adenovirus is used as an expression vector, the polypeptide or fusion protein coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 81:355-359). Specific initiation signals may also be required for efficient translation of inserted fusion protein coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

Expression vectors containing inserts of a gene encoding a polypeptide, peptide, protein, or fusion protein inhibitor of IL-9 can be identified by three general approaches: (a) nucleic acid hybridization, (b) presence or absence of “marker” gene functions, and (c) expression of inserted sequences. In the first approach, the presence of a gene encoding a polypeptide, peptide, protein, or a fusion protein inhibitor of IL-9 in an expression vector can be detected by nucleic acid hybridization using probes comprising sequences that are homologous to an inserted gene encoding the polypeptide, peptide, protein, or the fusion protein inhibitor of IL-9, respectively. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a nucleotide sequence encoding a polypeptide, peptide, protein, or fusion protein in the vector. For example, if the nucleotide sequence encoding the fusion protein is inserted within the marker gene sequence of the vector, recombinants containing the gene encoding the fusion protein insert can be identified by the absence of the marker gene function. In the third approach, recombinant expression vectors can be identified by assaying the gene product (e.g., fusion protein) expressed by the recombinant. Such assays can be based, for example, on the physical or functional properties of the fusion protein in in vitro assay systems, e.g., binding to an antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered fusion protein may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include, but are not limited to, CHO, VERO, BHK, Hela, COS, MDCK, 293, 3T3, W138, NS0, and in particular, neuronal cell lines such as, for example, SK-N-AS, SK-N-FI, SK-N-DZ human neuroblastomas (Sugimoto et al., 1984, J. Natl. Cancer Inst. 73: 51-57), SK-N-SH human neuroblastoma (Biochim. Biophys. Acta, 1982, 704: 450-460), Daoy human cerebellar medulloblastoma (He et al., 1992, Cancer Res. 52: 1144-1148) DBTRG-05MG glioblastoma cells (Kruse et al., 1992, In Vitro Cell. Dev. Biol. 28A: 609-614), IMR-32 human neuroblastoma (Cancer Res., 1970, 30: 2110-2118), 1321N1 human astrocytoma (Proc. Natl. Acad. Sci. USA, 1977, 74: 4816), MOG-G-CCM human astrocytoma (Br. J. Cancer, 1984, 49: 269), U87MG human glioblastoma-astrocytoma (Acta Pathol. Microbiol. Scand., 1968, 74: 465-486), A172 human glioblastoma (Olopade et al., 1992, Cancer Res. 52: 2523-2529), C6 rat glioma cells (Benda et al., 1968, Science 161: 370-371), Neuro-2a mouse neuroblastoma (Proc. Natl. Acad. Sci. USA, 1970, 65: 129-136), NB41A3 mouse neuroblastoma (Proc. Natl. Acad. Sci. USA, 1962, 48: 1184-1190), SCP sheep choroid plexus (Bolin et al., 1994, J. Virol. Methods 48: 211-221), G355-5, PG-4 Cat normal astrocyte (Haapala et al., 1985, J. Virol. 53: 827-833), Mpf ferret brain (Trowbridge et al., 1982, In Vitro 18: 952-960), and normal cell lines such as, for example, CTX TNA2 rat normal cortex brain (Radany et al., 1992, Proc. Natl. Acad. Sci. USA 89: 6467-6471) such as, for example, CRL7030 and Hs578Bst. Furthermore, different vector/host expression systems may effect processing reactions to different extents.

For long-term, high-yield production of recombinant polypeptide, peptide, protein, or fusion protein inhibitors of IL-9, stable expression is preferred. For example, cell lines which stably express a polypeptide, peptide, protein, or a fusion protein inhibitor of IL-9 may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express a polypeptide, peptide, protein, or a fusion protein that immunospecifically binds to an IL-9 polypeptide. Such engineered cell lines may be particularly useful in screening and evaluation of compounds that affect the activity of a polypeptide, peptide, protein, or fusion protein that immunospecifically binds IL-9.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk-, hgprt- or aprt-cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

Once a polypeptide, peptide, protein, or a fusion protein inhibitor of IL-9 has been produced by recombinant expression, it may be purified by any method known in the art for purification of a protein, for example, by chromatography (e.g., ion exchange, affinity, particularly by affinity for the specific antigen after Protein A, and sizing column chromatography), centrifugation, differential solubility, or by any other standard technique for the purification of proteins.

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification in their entirety.

### Examples

### Example 1

### IL-9 Receptor is Present in Human Non-Specific Colitis Tissue

Paraffin sections of resected human colon tissues (non-specific colitis) were stained using IL-9 receptor antibody. See figure la, which provides a non-specific colitis tissue sample positively stained for IL-9 receptor.

### Example 2

### IL-9 Receptor and IL-9 are Present in Human COPD Lung Tissue

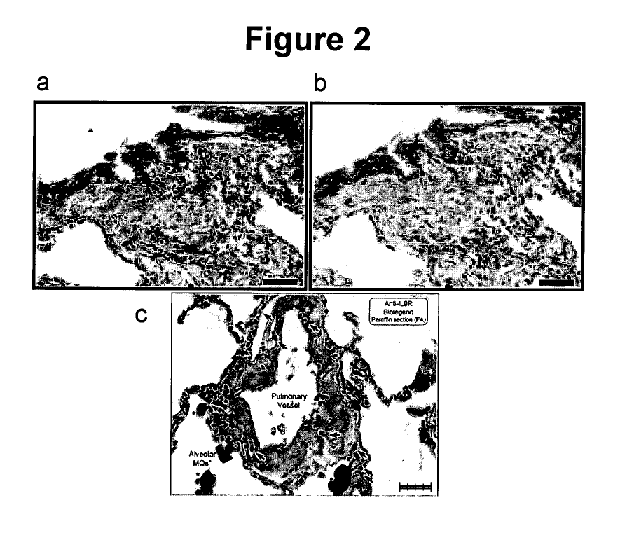

Paraffin sections of resected human COPD lung tissue were stained with IL-9 receptor antibody. See FIG. 2a, which provides the staining of COPD lung tissue with IL-9 receptor antibody. See also FIG. 2c, which shows that the distribution pattern of the IL-9 receptor positive cells closely matched that of tissue mast cells (mostly present in the walls of small airways) and pulmonary vessels.

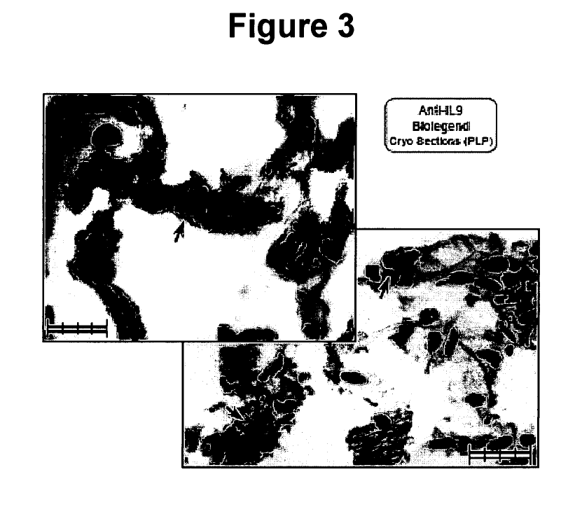

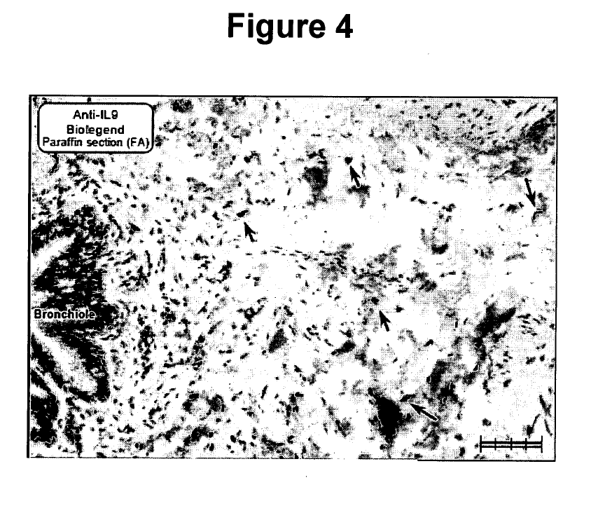

Cryo and paraffin sections of resected human COPD lung tissue were also stained with antibody specific for IL-9. Both the cryo (FIG. 3) and paraffin (FIG. 4) COPD lung samples exhibited IL-9 positive staining

### Example 3

### Anti IL-9 Antibody Reduces Mucus Production and Sub-Epithelial Collagen Deposition Induced by Chronic Allergen Challenge

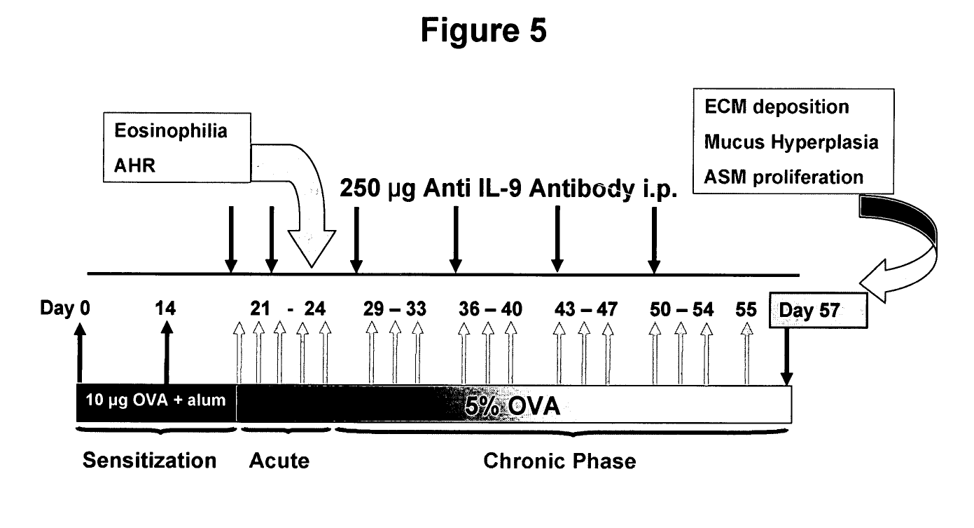

Chronic Challenge Protocol: Mice were immunized with OVA and chronically challenged as shown in FIG. 5.

Histology: Paraffin embedded sections (4 μm) were stained with haematoxylin/eosin (H & E) to evaluate general morphology, periodic acid-Schiff (PAS) to visualize goblet cells, and Sirius Red to assess matrix deposition.

Collagen Analysis: Total lung collagen content in lung tissue homogenates was measured by sircol assay. Peribronchial collagen deposition was determined using image analysis of Sirius Red stained lung sections to assess the density of collagen along 20 μm basement membrane.

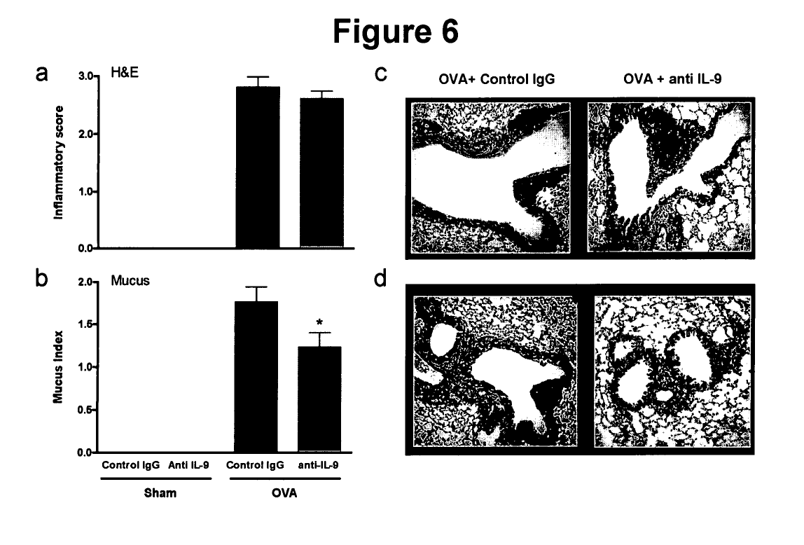

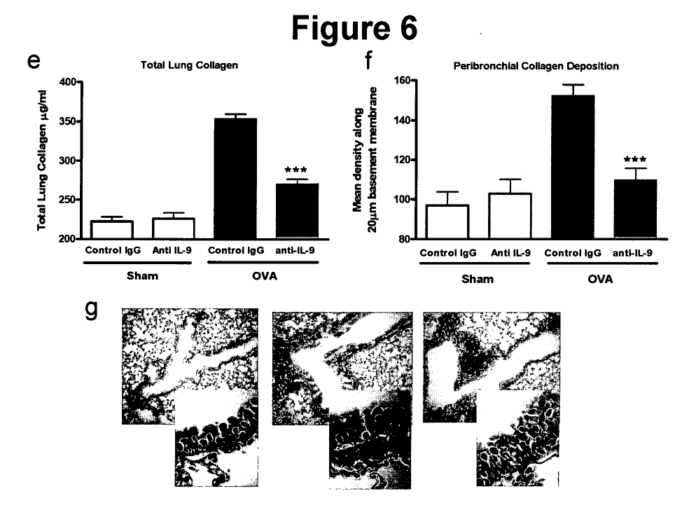

Results: Sham treated mice exhibited minimal H&E staining (inflammation), as expected in the absence of OVA treatment. In contrast, chronic allergen (OVA) challenge induced a significant increase in inflammation in mice, whether treated with control IgG or anti-IL-9 antibody. See FIGS. 6a and 6c.

To examine the ability of IL-9 antibody to inhibit airway remodeling in the chronic allergen challenge model, mucus secretion (a feature of airway remodeling) was examined in chronic allergen (OVA) challenged mice that received IgG control or IL-9 antibody. Mucus production in sham treated mice (n=16) and chronic allergen (OVA) treated mice (n=26-27) was assessed by PAS staining Anti IL-9 antibody treated OVA mice had significantly less mucus when compared to mice chronically challenged with OVA (FIGS. 6b and 6d). Results are expressed as mean±sem. The significant difference between OVA wild type mice and OVA anti IL-9 antibody treated mice is shown as *p<0.02. Thus, mucus production in chronic allergen challenged mice was attenuated by IL-9 antibody.

Subepithelial fibrosis is a distinctive feature of airway remodeling. It contributes to thickened airway walls due to the deposition of extracellular matrix proteins such as collagens, laminin and tenascin. Chronic allergen challenge induced a significant increase in total lung collagen (FIG. 6e) and peribronchial collagen deposition (FIG. 6f). Compare sham challenged mice to chronic allergen challenged mice in FIGS. 6e and 6f. Administration of IL-9 antibody to the chronic allergen challenged mice significantly reduced total lung collagen (FIG. 6e) and peribronchial collagen deposition (FIG. 6f). Significant differences between OVA wild type mice and OVA anti IL-9 antibody treated mice are shown as ***p<0.003-0.0001. Results are expressed as mean±sem (Sham group n=12; OVA mice n=19-20). In addition, peribronchial collagen deposition, as assessed on Sirius Red stained sections, was also markedly decreased (FIG. 6g). Thus, induction of collagen production in lungs by chronic allergen (OVA) challenge, like mucus production, was attenuated by IL-9 antibody administration.

### Example 4

### Anti IL-9 Antibody Reduces Levels of Pro-Fibrotic Mediators in Lung Tissue Following Chronic OVA Challenge

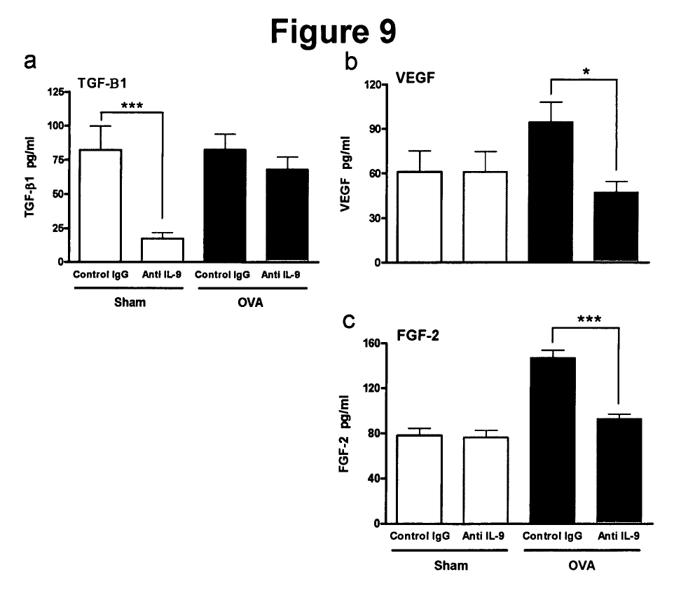

To further investigate the mechanisms by which IL-9 contributes to remodeling, levels of known pro-fibrotic mediators in lung tissue were determined. OVA challenge significantly induced levels of VEGF (FIG. 9b) and FGF-2 (FIG. 9c) in lung tissue compared to sham controls. These levels were reduced almost to baseline following IL-9 blockade (FIGS. 9b and 9c). Interestingly, anti IL-9 antibody had no effect on activated TGF-B1 levels following OVA challenge but did reduce constitutive levels of TGF-B1 in tissue when compared to controls (FIG. 9a). Thus, IL-9 antibody inhibited further mechanisms of remodeling, by decreasing levels of VEGF and FGF-2, in the chronic allergen (OVA) treated mice.

### Example 5

### Anti IL-9 Antibody Improves Lung Function Following Chronic Allergen Challenge

Assessment of Airway Hyperresponsiveness: AHR was determined as previously described (Bates et al., 2006, J. Appl. Physiol. 100: 500-506). At the start of the experiment, a standard lung volume history was established by delivering two deep lung inflations of 1 ml followed by 2 min of regular ventilation. Next, baseline recordings of all parameters (resistance, elastance, Newtonian resistance, tissue dampening, tissue elastance, and hysteresivity) were obtained. The mice were then exposed to PBS followed by sequentially increasing concentrations of methacholine (3.125, 12.5 & 50 mg/ml, 40 s aerosol exposure per dose and, recordings of all parameters were made every 10 s for 3 min).

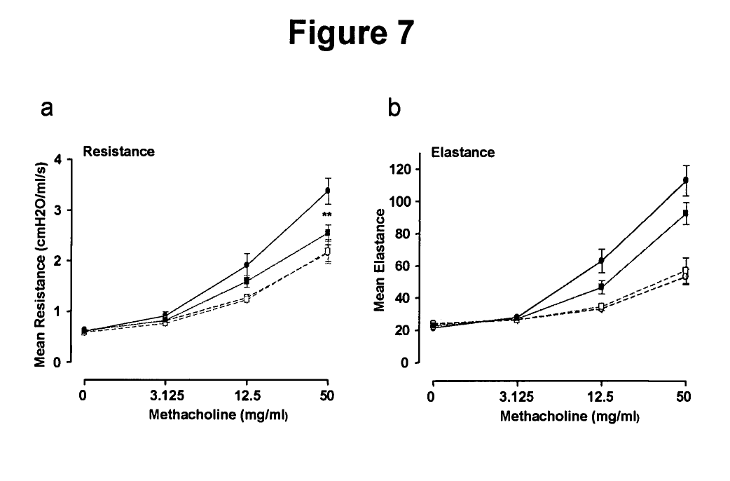

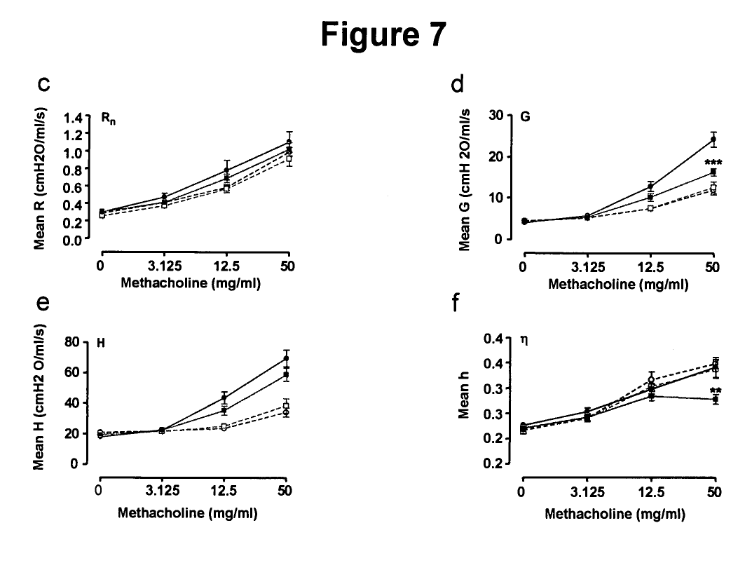

Results: Chronic allergen (OVA) challenge induced significant changes in lung function including increased resistance (FIG. 7a; ), increased elastance (FIG. 7b, ), increased Newtonian resistance (FIG. 7c; ), increased tissue dampening (FIG. 7d; ), increased tissue elastance (FIG. 7e; ), and increased hysteresivity (FIG. 7f; ) relative to sham challenge control (FIGS. 7a-7f; ∘) mice. Administration of IL-9 antibody to chronic allergen challenged mice significantly improved lung function across all parameters (FIG. 7a-7f; ▪). Significant differences between OVA+Control IgG vs OVA+anti IL-9 antibody treated mice are shown as **p<0.005 and ***p<0.001 for each of FIGS. 7a-7f.

### Example 6

### Anti IL-9 Antibody Significantly Attenuates Mast Cell Activation/Numbers in Tissue Following Chronic Allergen Challenge

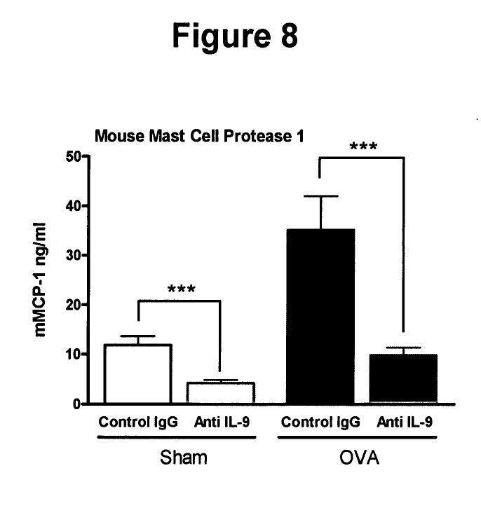

Mouse mast cell protease 1 levels were determined in serum following sham or chronic OVA challenge. OVA challenge significantly enhanced mMCP-1 levels compared to sham controls. The enhanced serum mMCP-1 levels in the OVA challenged mice were markedly reduced (to baseline) following IL-9 blockade. Interestingly, sham IL-9 treated mice also had reduced mMCP-1 levels when compared to controls. See FIG. 8.

### Example 7

### Treatment of Ulcerative Colitis with an IL-9 Antibody

A patient in need of treatment for ulcerative colitis will be administered IL-9 antibody 7F3com-2H2. The dose will be 9 kg/mg or 3 mg/kg administered intravenously or subcutaneously. The patient will be administered second and subsequent doses of the antibody at 2 week to once a month intervals.

1. A method of treating inflammatory bowel disease comprising:
administering an inhibitor of IL-9.

2. The method of claim 1 wherein the inflammatory bowel disease is ulcerative colitis, Crohn's disease, collagenous colitis, lymphocytic colitis, diversion colitis, or Behcet's syndrome.

3. The method of claim 2 wherein the inflammatory bowel disease is ulcerative colitis.

4. The method of claim 1 wherein the inhibitor of IL-9 is an antibody specific for IL-9.

5-8. (canceled)

9. The method of claim 4 wherein the antibody specific for IL-9 comprises the six CDRs of 7F3com-2H2.

10-19. (canceled)

20. A method of treating COPD comprising:
administering an inhibitor of IL-9.

21. The method of claim 20 wherein the inhibitor of IL-9 is an antibody specific for IL-9.

22. The method of claim 21 wherein the antibody specific for IL-9 is 4D4, 4D4H2-1 D11, 4D4com-XF-9, 4D4com-2F-9, 7F3, 71A10, 7F3 22D3, 7F3com-2H2, 7F3com-3H5, or 7F3com-3D4 or a fragment thereof.

23. The method of claim 22 wherein the antibody specific for IL-9 is 7F3com-2H2.

24. The method of claim 23 wherein the 7F3com-2H2 antibody is administered at a dose of 1 mg/kg, 3 mg/kg, 5 mg/kg, or 9 mg/kg.

25-27. (canceled)

28. The method of claim 21 wherein the antibody specific for IL-9 comprises a VH domain and a VL domain, wherein the VH domain comprises:
(a) a VH CDR1 having the amino acid sequence of the VH CDR1 of 7F3com-2H2 with less than 4 amino acid substitutions;
(b) a VH CDR2 having the amino acid sequence of the VH CDR2 of 7F3com-2H2 with less than 4 amino acid substitutions; and
(c) a VH CDR3 having the amino acid sequence of the VH CDR3 of 7F3com-2H2 with less than 4 amino acid substitutions; and
wherein the VL domain comprises:
(d) a VL CDR1 having the amino acid sequence of the VL CDR1 of 7F3com-2H2 with less than 4 amino acid substitutions;
(b) a VL CDR2 having the amino acid sequence of the VL CDR2 of 7F3com-2H2 with less than 4 amino acid substitutions; and
(c) a VL CDR3 having the amino acid sequence of the VL CDR3 of 7F3com-2H2 with less than 4 amino acid substitutions.

29-32. (canceled)

33. The method of claim 21 wherein the antibody specific for IL-9 is a monoclonal antibody, a human antibody, a humanized antibody, a chimeric antibody, a single Fv, an Fab fragment or an F(ab′) fragment.

34-35. (canceled)

36. A method of treating a fibrotic disorder comprising:
administering an inhibitor of IL-9.

37. The method of claim 36 wherein the inhibitor of IL-9 is an antibody specific for IL-9.

38. The method of claim 37 wherein the fibrotic disorder is liver cirrhosis, glomerulonephritis, pulmonary fibrosis, system fibrosis, rheumatoid arthritis, scleroderma, pancreatitis, and acute fibrosis.

39. The method of claim 37 wherein the antibody specific for IL-9 is 4D4, 4D4H2-1 D11, 4D4com-XF-9, 4D4com-2F-9, 7F3, 71A10, 7F3 22D3, 7F3com-2H2, 7F3com-3H5, or 7F3com-3D4 or a fragment thereof.

40. The method of claim 39 wherein the antibody specific for IL-9 is 7F3com-2H2.

41. (canceled)

42. The method of claim 37 wherein the antibody specific for IL-9 comprises the six CDRs of 7F3com-2H2.

43-44. (canceled)

45. The method of claim 37 wherein the antibody specific for IL-9 comprises a VH domain and a VL domain, wherein the VH domain comprises:
(a) a VH CDR1 having the amino acid sequence of the VH CDR1 of 7F3com-2H2 with less than 4 amino acid substitutions;
(b) a VH CDR2 having the amino acid sequence of the VH CDR2 of 7F3com-2H2 with less than 4 amino acid substitutions; and
(c) a VH CDR3 having the amino acid sequence of the VH CDR3 of 7F3com-2H2 with less than 4 amino acid substitutions; and
wherein the VL domain comprises:
(d) a VL CDR1 having the amino acid sequence of the VL CDR1 of 7F3com-2H2 with less than 4 amino acid substitutions;
(e) a VL CDR2 having the amino acid sequence of the VL CDR2 of 7F3com-2H2 with less than 4 amino acid substitutions; and
(f) a VL CDR3 having the amino acid sequence of the VL CDR3 of 7F3com-2H2 with less than 4 amino acid substitutions.

46-49. (canceled)

50. The method of claim 37 wherein the antibody specific for IL-9 or fragment thereof is a monoclonal antibody, a human antibody, a humanized antibody, a chimeric antibody, a single Fv, an Fab fragment or an F(ab′) fragment.

51-52. (canceled)


SEQUENCE LISTING (22 sequences)

SEQ ID NO: 1 - protein, 122 aa, artificial
  antibody 4D4 variable heavy chain
QVQLVQSGAEVKKPGASVKVSCKASGYTFTGYWIEWVRQAPGQGLEWMGEILPGSGTTNYNEKFKGRVTMTRDTSTSTVYMELSSLRSEDTAVYYCARADYYGSDYVKFDYWGQGTLVTVSS

SEQ ID NO: 2 - protein, 107 aa, artificial
  antibody 4D4 variable light chain
DIQMTQSPSSLSASVGDRVTITCKASQHVGTHVTWYQQKPGKAPKLLIYSTSYRYSGVPSRFSGSGSGTDFTLTISSLQPEDFATYYCQHFYSYPLTFGGGTKVEIK

SEQ ID NO: 3 - protein, 122 aa, artificial
  antibody 4D4H2-1 D11 variable heavy chain
QVQLVQSGAEVKKPGASVKVSCKASGYTFTGYWIEWVRQAPGQGLEWMGEWLPGSGTTNYNEKFKGRVTMTRDTSTSTVYMELSSLRSEDTAVYYCARADYYGSDYVKFDYWGQGTLVTVSS

SEQ ID NO: 4 - protein, 107 aa, artificial; identical to SEQ ID NO: 2
  antibody 4D4H2-1 D11 variable light chain

SEQ ID NO: 5 - protein, 122 aa, artificial
  antibody 4D4com-XF-9 variably heavy chain
QVQLVQSGAEVKKPGASVKVSCKASGYTFTYYWIEWVRQAPGQGLEWMGEWLPGSGTTNYNEKFKGRVTMTRDTSTSTVYMELSSLRSEDTAVYYCARADYYGSDHVKFDYWGQGTLVTVSS

SEQ ID NO: 6 - protein, 107 aa, artificial
  antibody 4D4com-XF-9 variably light chain
DIQMTQSPSSLSASVGDRVTITCLASQHVGTHVTWYQQKPGKAPKLLIYGTSYRYSGVPSRFSGSGSGTDFTLTISSLQPEDFATYYCQHFYDYPLTFGGGTKVEIK

SEQ ID NO: 7 - protein, 122 aa, artificial
  antibody 4D4com-2F-9 variable heavy chain
QVQLVQSGAEVKKPGASVKVSCKASGYTFTGYWIEWVRQAPGQGLEWMGEWLPGSGTTNYNEKFKGRVTMTRDTSTSTVYMELSSLRSEDTAVYYCARADYYGSDHVKFDYWGQGTLVTVSS

SEQ ID NO: 8 - protein, 107 aa, artificial
  antibody 4D4com-2F-9 variable light chain
DIQMTQSPSSLSASVGDRVTITCKASQHVGTHVTWYQQKPGKAPKLLIYGTSYRYSGVPSRFSGSGSGTDFTLTISSLQPEDFATYYCQHFYEYPLTFGGGTKVEIK

SEQ ID NO: 9 - protein, 122 aa, artificial
  antibody 7F3 variable heavy chain
QVQLVQSGAEVKKPGSSVKVSCKASGGTFSGYWIEWVRQAPGQGLEWMGEILPGSGTTNYNEKFKGRVTITADESTSTAYMELSSLRSEDTAVYYCARADYYGSDYVKFDYWGQGTLVTVSS

SEQ ID NO: 10 - protein, 107 aa, artificial
  antibody 7F3 variable light chain
DIQMTQSPSSLSASVGDRVTITCKASQHVGTHVTWYQQKPGKAPKLLIYSTSYRYSGVPSRFSGSGSGTDFTLTISSLQPEDFATYYCQQFYEYPLTFGGGTKVEIK

SEQ ID NO: 11 - protein, 122 aa, artificial
  antibody 71A10 variable heavy chain
QVQLVQSGAEVKKPGSSVKVSCKASGGTFSGYWIEWVRQAPGQGLEWMGEILPGSGTTNPNEKFKGRVTITADESTSTAYMELSSLRSEDTAVYYCARADYYGSDYVKFDYWGQGTLVTVSS

SEQ ID NO: 12 - protein, 107 aa, artificial; identical to SEQ ID NO: 10
  antibody 71A10 variable light chain

SEQ ID NO: 13 - protein, 122 aa, artificial; identical to SEQ ID NO: 9
  antibody 7F3 22D3 variable heavy chain

SEQ ID NO: 14 - protein, 107 aa, artificial
  antibody 7F3 22D3 variable light chain
DIQMTQSPSSLSASVGDRVTITCKASQHVGTHVTWYQQKPGKAPKLLIYGTSYRYSGVPSRFSGSGSGTDFTLTISSLQPEDFATYYCQQFYEYPLTFGGGTKVEIK

SEQ ID NO: 15 - protein, 122 aa, artificial
  antibody 7F3com2H2 variable heavy chain
QVQLVQSGAEVKKPGSSVKVSCKASGGTFSYYWIEWVRQAPGQGLEWMGEILPGSGTTNPNEKFKGRVTITADESTSTAYMELSSLRSEDTAVYYCARADYYGSDYVKFDYWGQGTLVTVSS

SEQ ID NO: 16 - protein, 107 aa, artificial
  antibody 7F3com2H2 variable light chain
DIQMTQSPSSLSASVGDRVTITCKASQHVITHVTWYQQKPGKAPKLLIYGTSYSYSGVPSRFSGSGSGTDFTLTISSLQPEDFATYYCQQFYEYPLTFGGGTKVEIK

SEQ ID NO: 17 - protein, 122 aa, artificial; identical to SEQ ID NO: 11
  antibody 7F3com3H5 variable heavy chain

SEQ ID NO: 18 - protein, 107 aa, artificial; identical to SEQ ID NO: 14
  antibody 7F3com3H5 variable light chain

SEQ ID NO: 19 - protein, 122 aa, artificial; identical to SEQ ID NO: 15
  antibody 7F3com3D4 variable heavy chain

SEQ ID NO: 20 - protein, 107 aa, artificial
  antibody 7F3com3D4 variable light chain
DIQMTQSPSSLSASVGDRVTITCKASQHVITHVTWYQQKPGKAPKLLIYGTSYRYSGVPSRFSGSGSGTDFTLTISSLQPEDFATYYCQQFYEYPLTFGGGTKVEIK

SEQ ID NO: 21 - DNA, 366 bp, artificial
  DNA encoding antibody 7F3com2H2 variable heavy chain
caggtgcagctggtgcagtctggggctgaggtgaagaagcctgggtcctcagtgaaggtttcctgcaaggcatctggaggcaccttcagctattactggatagagtgggtgcgacaggcccctggacaagggcttgagtggatgggagagattttacctggaagtggtactactaacccgaatgagaagttcaagggcagagtcaccattaccgcggacgaatccacgagcacagcctacatggagctgagcagcctgagatctgaggacacggccgtgtattactgtgcgagagcggattactacggtagtgattacgtcaagtttgactactggggccaaggaaccctggtcaccgtctcctca

SEQ ID NO: 22 - DNA, 321 bp, artificial
  DNA encoding antibody 7F3com2H2 variable light chain
gacatccagatgacccagtctccatcctccctgtctgcatctgtaggagacagagtcaccatcacttgcaaggcaagtcagcatgtgattactcatgtaacctggtatcagcagaaaccagggaaagcccctaagctcctgatctatgggacatcctacagctacagtggggtcccatcaaggttcagtggcagtggatatgggacagatttcactctcaccatcagcagtctgcaacctgaagattttgcaacttattactgtcagcaattttacgagtatcctctcacgttcggcggagggaccaaggtggagatcaaa